SEQUENCE LISTING (8 sequences)

SEQ ID NO: 1 - DNA, 48 bp, synthetic construct
  source 1..48: mol_type = other DNA
atatccctcgccgaatcctagactcaaagtagtctaggattcggcgag

SEQ ID NO: 2 - DNA, 48 bp, synthetic construct
  source 1..48: mol_type = other DNA
agtctaggattcggcgagggatatctcgccgaatcctagactactttg

SEQ ID NO: 3 - DNA, 47 bp, synthetic construct
  source 1..47: mol_type = other DNA
agtctaggattcggcgagggatattttttctcttggaaagaaagtgc

SEQ ID NO: 4 - DNA, 14 bp, synthetic construct
  source 1..14: mol_type = other DNA
tttttgcactttct

SEQ ID NO: 5 - DNA, 29 bp, synthetic construct
  source 1..29: mol_type = other DNA
agtctaggattcggcgagggatatttttt

SEQ ID NO: 6 - DNA, 14 bp, synthetic construct
  source 1..14: mol_type = other DNA
tttttttccaagag

SEQ ID NO: 7 - DNA, 14 bp, synthetic construct
  source 1..14: mol_type = other DNA
tttttgcactttct

SEQ ID NO: 8 - DNA, 29 bp, synthetic construct
  source 1..29: mol_type = other DNA
agtctaggattcggcgagggatatttttt

(19) United States
(12) Patent Application Publication
(10) Pub. No.: US 2025/0277787 A1
(43) Pub. Date: Sep. 4, 2025

(54) ENHANCED LATERAL FLOW ASSAYS AND DEVICES FOR DETECTING ANALYTES IN BLOOD SAMPLES

(71) Applicant: Intelligent Optical Systems, Inc., Torrance, CA (US)

(72) Inventors: Trong Nguyen, Garden Grove, CA (US); Manal Beshay, Torrance, CA (US); Maria Ortega, Bell Gardens, CA (US)

(21) Appl. No.: 18/698,808

(22) PCT Filed: Oct. 7, 2022
(86) PCT No.: PCT/US2022/046124

§ 371 Date: Apr. 5, 2024

Related U.S. Application Data
(60) Provisional application No. 63/253,518, filed on Oct. 7, 2021.
(60) Provisional application No. 63/270,501, filed on Oct. 21, 2021.

(51) Int. Cl. G01N 33/543; G01N 33/58; G01N 33/94

(52) U.S. Cl. CPC G01N 33/54388; G01N 33/582; G01N 33/9493

(57) ABSTRACT

A lateral flow device for detecting an analyte in a sample, the device comprising: a sample region; a conjugate region comprising: a detection agent that binds an analyte; a hybridization initiator coupled to the detection agent; a plurality of nucleic acid probes, wherein the plurality of nucleic acid probes comprises a first nucleic acid probe and a second nucleic acid probe, and a third nucleic acid probe; wherein the first nucleic acid probe hybridizes to a portion of the hybridization initiator and a portion of the second nucleic acid probe; wherein the second nucleic acid probe hybridizes to a portion of the first nucleic acid probe and a portion of the third nucleic acid probe; and wherein the third nucleic acid probe hybridizes to a portion of the second nucleic acid probe; and a detection region comprising: a capture agent immobilized on the detection region.

### CLAIM OF PRIORITY

This application is a National Stage Application filed under 35 U.S.C. § 371 of PCT International Application No. PCT/US2022/046124, filed Oct. 7, 2022, which claims the benefit of U.S. Provisional Application Nos. 63/253,518, filed on Oct. 7, 2021, and 63/270,501, filed on Oct. 21, 2021, all of which are incorporated by reference herein in their entirety.

### FEDERALLY SPONSORED RESEARCH OR DEVELOPMENT

This invention was made with Government support under Grant No. W81XWH-20-C-0044 awarded by the Department of Defense. The Government has certain rights in the invention.

### INCORPORATION BY REFERENCE

This application contains a Sequence Listing that has been submitted electronically as an XML file named 54785-0002US1_ST26_SL.XML.” The XML file, created on Sep. 12, 2024, is 8,197 bytes in size. The material in the XML file is hereby incorporated by reference in its entirety.

### FIELD OF THE INVENTION

The subject matter disclosed herein generally relates to lateral flow assays and devices for detecting analytes in a blood sample.

### BACKGROUND

Drugs having a narrow therapeutic index are drugs in which a small difference in dose or blood concentration may lead to serious therapeutic failures and/or adverse drug reactions that are life-threatening or result in persistent or significant disability or incapacity. Current drug monitoring methods involve hospitalization and/or frequent phlebotomy that contributes to infrequent monitoring and suboptimal drug peak and trough levels. Thus, there is a need for drug monitoring methods and devices that are patient-friendly, reliable, and accurate in a diversity of care settings.

### SUMMARY

The present disclosure is based, at least in part, on the development of enhanced lateral flow assay (ELFA) methods and devices for detecting analytes in a blood sample that provide rapid results (e.g., quantitative results obtained in 15 to 20 minutes after sampling) with high sensitivity (e.g., detection of 2 ng/ml of tacrolimus) using blood samples obtained from minimally invasive techniques (e.g., small blood sample volumes obtained using a fingerstick).

A first general aspect of the present disclosure provides a lateral flow device for detecting an analyte in a sample, the device comprising:
a sample region;
a conjugate region comprising a detection agent that binds an analyte, wherein the detection agent is labeled with a first detectable label; a hybridization initiator coupled to the detection agent or the first detectable label; a plurality of nucleic acid probes, each of which is labeled with at least one second detectable label, wherein the plurality of nucleic acid probes comprises a first nucleic acid probe and a second nucleic acid probe, and a third nucleic acid probe; wherein the first nucleic acid probe hybridizes to a portion of the hybridization initiator and a portion of the second nucleic acid probe; wherein the second nucleic acid probe hybridizes to a portion of the first nucleic acid probe and a portion of the third nucleic acid probe; and wherein the third nucleic acid probe hybridizes to a portion of the second nucleic acid probe; and
a detection region comprising a capture agent that binds the analyte, wherein the capture agent is immobilized on the detection region.

Implementations of the first general aspect may include one or more of the following features.

In some embodiments, the detection agent comprises an antibody that binds the analyte. In some embodiments, a concentration of the detection agent applied on the conjugate region is in a range of 0.01 μg/μL to 0.1 μg/μL.

In some embodiments, the capture agent comprises an antibody that binds the analyte. In some embodiments, a concentration of the capture agent applied on the detection region is in a range of 0.5 mg/mL to 5 mg/mL.

In some embodiments, the detection agent and the capture agent comprise the same antibody. In some embodiments, the detection agent and the capture agent comprise different antibodies.

In some embodiments, the hybridization initiator comprises DNA, RNA, or both. In some embodiments, a concentration of the hybridization initiator applied on the conjugate region is in a range of 5 nM to 40 nM.

In some embodiments, each nucleic acid probe in the plurality of nucleic acid probes comprises DNA, RNA, or both. In some embodiments, a concentration of each nucleic acid probe in the plurality of nucleic acid probes applied on the conjugate region is in a range of 5 nM to 40 nM.

In some embodiments, the hybridization initiator and the first detectable label are conjugated to members of a receptor-ligand pair that mediates attachment of the hybridization initiator to the first detectable label. In some embodiments, the nucleic acid probe and the second detectable label are conjugated to members of a receptor-ligand pair that mediates attachment of the nucleic acid probe to the second detectable label. In some embodiments, the receptor-ligand pair comprises biotin and avidin. In some embodiments, the avidin comprises streptavidin.

In some embodiments, the first detectable label and the second detectable label are visually detectable labels. In some embodiments, the first detectable label comprises europium, colloidal gold, phycoerythrin, fluorescein, green fluorescent protein, quantum dots, rhodamine, or a combination thereof. In some embodiments, the second detectable label comprises europium, colloidal gold, phycoerythrin, fluorescein, green fluorescent protein, quantum dots, rhodamine, or a combination thereof. In some embodiments, the first detectable label and the second detectable label are the same. In some embodiments, the first detectable label and the second detectable label comprise europium.

In some embodiments, the analyte is a drug. In some embodiments, the drug comprises an immunosuppressant, an antibiotic, an anticoagulant, an antidepressant, a bronchodilator, an anticonvulsant, an antiarrhythmic, an aminoglycoside, or a combination thereof. In some embodiments, the immunosuppressant comprises tacrolimus, ascomycin, everolimus, pimecrolimus, ridaforolimus, temsirolimus, umirolimus, ascomycin, sirolimus, cyclosporine, zonotarolimus, or a combination thereof.

In some embodiments, the detection region comprises a test region and a control region. In some embodiments, the test region comprises the capture agent and the control region comprises the control agent that binds to the detection agent.

In some embodiments, the device further comprises an absorbent region for absorbing excess sample and maintaining a lateral flow along the detection region.

In some embodiments, the conjugate region is between the sample region and the detection region.

In some embodiments, the device is configured to allow the sample to flow from the sample region to the detection region.

A second general aspect of the present disclosure provides a sample buffer comprising a surfactant; a salt; a sugar; and a protein.

Implementations of the second general aspect may include one or more of the following features.

In some embodiments, the surfactant comprises sodium dodecyl sulfate, polysorbate, octylphenol ethoxylate, 2-[4-(2,4,4-trimethylpentan-2-yl)phenoxy]ethanol, sodium deoxy cholate, 3-[N,N-Dimethyl(3-myristoylaminopropyl)ammonio]propanesulfonate, polyoxyethylene lauryl ether, polyethylene glycol, 4-(1,1,3,3-tetramethylbutyl)phenyl-polyethylene glycol, t-octylphenoxypolyethoxyethanol, or a combination thereof.

In some embodiments, the salt comprises a sodium salt, a potassium salt, a phosphate salt, a zinc salt, or a combination thereof.

In some embodiments, the sodium salt comprises sodium acetate, sodium carbonate, sodium bicarbonate, sodium chloride, sodium citrate, sodium phosphate monobasic, sodium phosphate dibasic, sodium sulfate, sodium thiosulfate, sodium triphosphate, or a combination thereof.

In some embodiments, the sugar comprises trehalose, sucrose, glucose, or a combination thereof.

In some embodiments, the protein comprises gelatin, albumin, casein, or a combination thereof.

In some embodiments, the surfactant comprises polysorbate 20, polyethylene glycol having an average molecular weight of 20,000 daltons (PEG 20), and 4-(1,1,3,3-tetramethylbutyl)phenyl-polyethylene glycol; the sodium salt comprises sodium chloride; the sugar comprises trehalose and sucrose; and the protein comprises casein.

In some embodiments, the polysorbate 20 is present in a total amount of about 0.05 wt % to about 10 wt % of the buffer; the 4-(1,1,3,3-tetramethylbutyl)phenyl-polyethylene glycol is present in a total amount of about 0.001 wt % to about 4 wt % of the buffer; the sodium chloride is present at a concentration of about 300 mM to about 1500 mM; the PEG20 is present in a total amount of about 0.05 wt % to 10 wt % of the buffer; the trehalose is present in a total amount of about 1 wt % to 30 wt % of the buffer; the sucrose is present in a total amount of about 1 wt % to 30 wt % of the buffer; and the casein is present in a total amount of about 0.1 wt % to 10 wt % of the buffer.

In some embodiments, the polysorbate 20 is present in a total amount of about 0.05 wt % to about 2 wt % of the buffer; the 4-(1,1,3,3-tetramethylbutyl)phenyl-polyethylene glycol is present in a total amount of about 0.05 wt % to about 1 wt % of the buffer; the sodium chloride is present at a concentration of about 2 mM to about 500 mM; the PEG20 is present in a total amount of about 0.05 wt % to 1 wt % of the buffer; the trehalose is present in a total amount of about 1 wt % to 5 wt % of the buffer; the sucrose is present in a total amount of about 1 wt % to 5 wt % of the buffer; and the casein is present in a total amount of about 0.1 wt % to 1 wt % of the buffer.

In some embodiments, the buffer further comprises polyoxyethylene (23) lauryl ether. In some embodiments, the polyoxyethylene (23) lauryl ether is present in a total amount of about 0.001 wt % to about 5 wt % of the buffer. In some embodiments, the polyoxyethylene (23) lauryl ether is present in a total amount of about 0.5 wt % to about 2 wt % of the buffer.

In some embodiments, the buffer further comprises potassium dihydrogen phosphate. In some embodiments, the potassium dihydrogen phosphate is present at a concentration of about 1 mM to about 500 mM. In some embodiments, the potassium dihydrogen phosphate is present at a concentration of about 1 mM to about 5 mM.

In some embodiments, the buffer further comprises potassium chloride. In some embodiments, the potassium chloride is present at a concentration of about 1 mM to about 200 mM. In some embodiments, the potassium chloride is present at a concentration of about 3 mM to about 30 mM.

In some embodiments, the buffer further comprises trisodium citrate. In some embodiments, the trisodium citrate is present at a concentration of about 5 mM to about 500 mM. In some embodiments, the trisodium citrate is present at a concentration of about 10 mM to about 100 mM.

In some embodiments, the buffer further comprises sodium dihydrogen phosphate. In some embodiments, the sodium dihydrogen phosphate is present at a concentration of about 1 mM to about 100 mM. In some embodiments, the sodium dihydrogen phosphate is present at a concentration of about 5 mM to about 10 mM.

In some embodiments, the buffer further comprises sodium deoxycholate. In some embodiments, the sodium deoxycholate is present in a total amount of about 0.02 wt % to about 15 wt % of the buffer. In some embodiments, the sodium deoxycholate is present in a total amount of about 0.5 wt % to about 1 wt % of the buffer.

In some embodiments, the buffer further comprises sodium dodecyl sulfate. In some embodiments, the sodium dodecyl sulfate is present in a total amount of about 0.5 wt % to about 5 wt % of the buffer. In some embodiments, sodium dodecyl sulfate is present in a total amount of about 0.5 wt % to about 2 wt % of the buffer.

In some embodiments, the buffer further comprises t-octylphenoxy polyethoxyethanol. In some embodiments, the t-octylphenoxypolyethoxyethanol is present in a total amount of about 0.001 wt % to about 4 wt % of the buffer. In some embodiments, t-octylphenoxy polyethoxyethanol is present in a total amount of about 0.5 wt % to about 2 wt % of the buffer.

In some embodiments, the buffer further comprises an organic solvent. In some embodiments, the organic solvent comprises methanol, acetone, acetonitrile, isopropanol, dichloromethane, ethanol, or a combination thereof. In some embodiments, the organic solvent comprises acetonitrile. In some embodiments, the acetonitrile is present in a total amount of about 1 wt % to about 50 wt % of the buffer. In some embodiments, the acetonitrile is present in a total amount of about 5 wt % to about 30 wt % of the buffer.

A third general aspect of the present disclosure provides a method for detecting an analyte in a sample, the method comprising:
contacting the sample region of the device of the first general aspect with a sample;
transporting at least a portion of the sample through the conjugate region and the detection region via capillary flow;
binding an analyte to the detection agent bound to the first detectable label and at least one second detectable label to produce a complex comprising the detection agent and the analyte;
binding the complex comprising the detection agent and the analyte to the capture agent; and
detecting a signal from the first detectable label and the at least one second detectable label in the detection region.

Implementations of the third general aspect may include one or more of the following features.

In some embodiments, the method further comprises determining a level of the analyte in the sample based on the level of the signal.

In some embodiments, detecting the signal is performed by a human eye. In some embodiments, detecting the signal is performed by placing the device in a signal detection reader.

In some embodiments, the method further comprises combining the sample with the sample buffer of the second general aspect prior to contacting the sample with the device. In some embodiments, the sample comprises cells and the sample is incubated with the sample buffer for a time sufficient to release the analyte from the cells. In some embodiments, the sample is incubated with the sample buffer for 1 to 15 minutes prior to contacting the sample region with the sample.

In some embodiments, the sample is a biological sample obtained from a subject. In some embodiments, the biological sample is a whole blood sample, a serum sample, or a plasma sample. In some embodiments, the biological sample is a whole blood sample collected via a fingerstick device. In some embodiments, a volume of the whole blood sample is in a range of 5 μL to 100 μL. In some embodiments, the blood sample is obtained by the subject. In some embodiments, the subject has had or is going to have a tissue or organ transplant. In some embodiments, the subject is a human patient.

In some embodiments, the analyte is a drug. In some embodiments, the drug comprises an immunosuppressant, an antibiotic, an anticoagulant, an antidepressant, a bronchodilator, an anticonvulsant, an antiarrhythmic, an aminoglycoside, or a combination thereof. In some embodiments, the immunosuppressant comprises tacrolimus, ascomycin, everolimus, pimecrolimus, ridaforolimus, temsirolimus, umirolimus, ascomycin, sirolimus, cyclosporine, zonotarolimus, or a combination thereof.

A fourth general aspect of the present disclosure provides a method of selecting a dose of an immunosuppressant for a subject comprising:
obtaining a blood sample from a subject who has been administered an immunosuppressant at a first dose;
providing the blood sample to the sample region of the device of the first general aspect;
advancing at least a portion of the blood sample through the conjugate region and the detection region via capillary flow;
binding an immunosuppressant to the detection agent coupled to the first detectable label and at least one second detectable label to produce a complex comprising the detection agent and the immunosuppressant;
binding the complex comprising the detection agent and the immunosuppressant to the capture agent; and
detecting a signal from the first detectable label and the at least one second detectable label in the detection region.

Implementations of the fouth general aspect may include one or more of the following features.

In some embodiments, the method further comprises determining a level of the immunosuppressant in the sample based on the level of the signal.

In some embodiments, the method further comprises determining a second dose of the immunosuppressant for the subject based on the level of the immunosuppressant in the sample.

In some embodiments, obtaining the blood sample comprises using a fingerstick device.

In some embodiments, contacting the blood sample with the sample region comprises applying the blood directly to the sample region.

In some embodiments, the method further comprises combining the blood sample with the sample buffer of the second general aspect prior to contacting the blood sample with the device. In some embodiments, the blood sample is incubated with the sample buffer for a time sufficient to release the immunosuppressant from the blood cells. In some embodiments, the blood sample is incubated with the sample buffer for 1 to 15 minutes prior to contacting the blood sample with the device.

In some embodiments, the method further comprises administering the immunosuppressant at a second dose based on the amount of the immunosuppressant in the blood sample. In some embodiments, the second dose is higher or lower than the first dose.

A fifth general aspect of the present disclosure provides a kit comprising a device of the first general aspect and a sample buffer of the second general aspect.

Implementations of the fifth general aspect may include one or more of the following features.

In some embodiments, the kit further comprises a fingerstick device.

In some embodiments, the kit further comprises a signal detection reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, scientific articles, published patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1A includes a schematic depiction showing hybridization chain reaction (HCR) components in a system with three nucleic acid probes. Letters marked with * are complementary to the corresponding unmarked letter. The nucleic acid probes (P1, P2, and P3) are shown labeled with detectable labels (circles) at the 5′ and 3′ ends. The nucleic acid probes form hairpins in the absence of initiator.

FIG. 1B includes a schematic depiction showing hybridization of the initiator to P1, which uncoils P1 from its hairpin structure to form a nicked double helix with a sticky end (c and b) that is complementary to P2 (c* and b*).

FIG. 1C includes a schematic depiction showing hybridization of the nucleic acid probes into a nicked double helix that concentrates the detectable labels and dramatically enhances the detectable signal.

FIG. 1D includes a schematic depiction showing hybridization chain reaction (HCR) components in a system with two nucleic acid probes. Letters marked with * are complementary to the corresponding unmarked letter. The nucleic acid probes (P1 and P2) are shown labeled with detectable labels (circles) at the 5′ and 3′ ends. The nucleic acid probes form hairpins in the absence of initiator. Hybridization of the initiator to P1 and hybridization between alternating P1 and P2 hairpins forms a nicked double helix that concentrates the detectable labels and dramatically enhances the detectable signal.

FIG. 2 includes a schematic depiction of an exemplary enhanced lateral flow assay (ELFA) device for detecting an analyte.

FIG. 3 includes a schematic depiction of an exemplary ELFA device for detecting multiple analytes in a sample. The ELFA device includes three different capture agents that are deposited in three separate test zones for detecting the immunosuppressant tacrolimus and the inflammation markers MMP3 and IL6, respectively. Detection of the analytes at the test zones involves use of the HCR system described herein, which concentrates the detectable labels at the test zones and dramatically enhances the detectable signal.

FIG. 4 includes an image of an exemplary ELFA device, a commercial reader for detecting assay results, a mobile phone on which the results can be displayed.

FIG. 5 includes results from testing detection of tacrolimus using various tacrolimus antibody pairs in capture and detector configurations in the ELFA platform. Five running buffers were used to test each antibody pair.

FIG. 6A includes images of ELFA devices comprising 0.25, 0.5, or 1 μL of conjugated detector antibody that were used to test detection of 0 and 25 ng/ml of Tac spiked in a 100 μL sample.

FIG. 6B includes a graph of the ratio of the signal from the capture line and the control line (capture line:control line) plotted against the amount of tacrolimus (ng/ml) in the sample.

FIG. 7A includes a graph of the standard calibration curve (SCC) for detection of tacrolimus (0 ng/mL to 48 ng/ml of tacrolimus). Data was recorded at 10, 20, and 30 minute endpoints (time from applying the sample to reading). Data for the 10 minute endpoint is shown in FIG. 7B.

FIG. 7B includes images of ELFA devices (top panel) and nitrocellulose membranes (bottom left panel) used to generate the SCC curve (bottom right panel) for detection of tacrolimus at the 10 minute endpoint.

FIG. 8A includes images of ELFA devices used to detect tacrolimus in plasma samples spiked with 0, 1.25, 2.5, 5, 10, 15, 20, and 30 ng/ml of tacrolimus. Data was recorded at 10 minute and 20 minute endpoints.

FIG. 8B includes images of ELFA devices used to test assay reproducibility with plasma samples spiked with tacrolimus (n=5).

FIG. 8C includes graphs of the signal (line peak height to capture/control ratio measurements) plotted against the amount of tacrolimus in the sample. Left panel shows data for samples containing 0 ng/mL to 35 ng/ml of tacrolimus. Right panel shows data for samples containing 0 ng/ml to 10 ng/mL.

FIG. 8D includes results from ELFA repeatability testing in plasma spiked with tacrolimus (n=5), comparing capture line peak height to measured capture/control ratio. The ratio measurement resulted in a better assay correction for background and flow inconsistency.

FIG. 9A includes images of ELFA devices stored at room temperature for up to 374 days and used to detect tacrolimus in spiked samples.

FIG. 9B includes a graph of detection of tacrolimus by ELFA devices after storage.

FIG. 10 includes a graph from testing three ratios of detector antibody:latex beads (10, 20, and 40 μg/100 μL) for aggregation at the sample pads. Samples included tacrolimus at 0, 5, or 10 ng/ml.

FIG. 11A includes images showing detection of tacrolimus in whole blood samples using the ELFA device described herein.

FIG. 11B includes a graph of the relative readout signal vs. the concentration of tacrolimus in whole blood samples.

FIG. 11C includes a table showing data collected from evaluating detection of tacrolimus in whole blood using ELFA methods and devices described herein.

FIG. 12 includes data from preliminary testing of whole blood samples on the ELFA platform integrated with red-blood-cell filtration pads.

FIG. 13 includes data from testing of the ELFA platform for detection of tacrolimus in whole blood samples with or without sample buffer treatment.

FIG. 14A includes a graph showing data from calibration of the ELFA platform using whole blood samples spiked with various concentrations of tacrolimus.

FIG. 14B includes a table showing data from calibration of the ELFA platform using whole blood samples spiked with various concentrations of tacrolimus.

FIG. 15A includes a graph showing Pearson's correlation for detection of tacrolimus in blood samples spiked with tacrolimus using the ELFA platform and standard LC-MS methods.

FIG. 15B includes a graph showing Bland-Altman analysis for detection of tacrolimus in blood samples spiked with tacrolimus using the ELFA platform and standard LC-MS methods.

FIG. 16 includes a graph showing intra-and inter-assay test data.

FIG. 17 includes a graph showing results for intra-assay repeatability testing performed on a single human whole blood sample spiked with 4 ng/ml to 24 ng/ml of tacrolimus.

FIG. 18 includes a graph showing results for inter-assay repeatability testing performed on a single human whole blood sample spiked with 0 ng/ml to 24 ng/ml of tacrolimus.

FIG. 19A includes a graph showing results for testing detection of tacrolimus in the ELFA platform using Buffers 1-6.

FIG. 19B includes a graph showing results for testing detection of tacrolimus in the ELFA platform using Buffers 5 and 7-11.

FIG. 19C includes a graph showing results for testing detection of tacrolimus in the ELFA platform using Buffers 11-13.

FIG. 19D includes a graph showing results for testing detection of tacrolimus in the ELFA platform using Buffers 11 and 14-16.

### DETAILED DESCRIPTION

The present disclosure is based, at least in part, on the development of enhanced lateral flow assay (ELFA) methods and devices for detecting an analyte. The ELFA methods and devices are designed for specifically detecting a presence of an analyte such as a drug (e.g., an immunosuppressant such as tacrolimus) and/or measuring a level of the analyte in a blood sample from a subject.

### I. Components for Use in the Enhanced Lateral Flow Assay (ELFA)

The ELFA methods and devices disclosed herein involve a detection agent and a capture agent that bind to a target analyte. The detection agent is conjugated to a detectable label. The ELFA methods and devices disclosed herein also involve a hybridization initiator and a plurality of nucleic acid probes for producing a detectable signal indicative of a presence or a level of an analyte in a sample. The hybridization initiator is conjugated to the detection agent or to the detectable label.

### (a) Detection and Capture Agents

The ELFA methods and devices disclosed herein involve a sandwich assay format in which a target analyte is bound by a detection agent and an immobilized capture agent. A detection agent is a molecule (e.g., a protein or a fragment thereof) that specifically binds a target analyte. A capture agent is a molecule (e.g., a protein or a fragment thereof) that specifically binds a target analyte.

A molecule is said to exhibit “specific binding” if it reacts more frequently, more rapidly, with greater duration and/or with greater affinity with a particular target (e.g., those disclosed herein) than it does with alternative targets. For example, a molecule that specifically binds tacrolimus would react more frequently, more rapidly, with greater duration and/or with greater affinity to tacrolimus as compared to an alternative target such as another immunosuppressant (e.g., everolimus, sirolimus, cyclosporine). “Specific binding” or “preferential binding” does not necessarily require (although it can include) exclusive binding.

The detection agent and/or the capture agent can be an antibody that binds a target analyte (e.g., an immunosuppressant such as tacrolimus). As used herein, the term “antibody” refers to a protein that includes at least one immunoglobulin variable domain or immunoglobulin variable domain sequence. For example, an antibody can include a heavy (H) chain variable region (VH) and a light (L) chain variable region (VL). In another example, an antibody includes two heavy chain variable regions and two light chain variable regions. The term “antibody” encompasses full-length antibodies and antigen-binding fragments of antibodies (e.g., single chain antibodies, Fab and sFab fragments, F(ab′)₂, Fd fragments, Fv fragments, scFv, and domain antibodies (dAb) fragments. An antibody can have the structural features of IgA, IgG, IgE, IgD, IgM (as well as subtypes thereof).

In some embodiments, an antibody that binds a target analyte (e.g., an immunosuppressant such as tacrolimus) may specifically bind to the target analyte, for example, an epitope of the target analyte. An antibody that “specifically binds” to an antigen or an epitope is a term well understood in the art, and methods to determine such specific binding are also well known in the art. An antibody is said to exhibit “specific binding” if it reacts or associates more frequently, more rapidly, with greater duration and/or with greater affinity with a particular target than it does with alternative targets. An antibody “specifically binds” to a target antigen or epitope if it binds with greater affinity, avidity, more readily. and/or with greater duration than it binds to other molecules. For example, an antibody that specifically (or preferentially) binds to an antigen (e.g., an immunosuppressant such as tacrolimus) or an antigenic epitope therein is an antibody that binds this target antigen with greater affinity, avidity, more readily, and/or with greater duration than it binds to other antigens or other epitopes in the same target antigen. It is also understood that an antibody that specifically binds to a first target antigen may or may not specifically or preferentially bind to a second target antigen. As such, “specific binding” or “preferential binding” does not necessarily require (although it can include) exclusive binding. Generally, but not necessarily. reference to binding means preferential binding. In some examples, an antibody that “specifically binds” to a target antigen or an epitope thereof may not bind to other antigens or other epitopes in the same target antigen.

An antibody that binds a target analyte can be either monoclonal or polyclonal. A “monoclonal antibody” refers to a homogenous antibody population and a “polyclonal antibody” refers to a heterogeneous antibody population. These two terms do not limit the source of an antibody or the manner in which it is made. An antibody for use in methods disclosed herein can be commercially obtained or made by any method known in the art, e.g., conventional hybridoma technology.

In some examples, the detection agent and/or the capture agent is an antibody or fragment thereof that binds a target analyte (e.g., an immunosuppressant such as tacrolimus). Example anti-tacrolimus antibodies that bind to tacrolimus are provided in clone 14H04 (see, e.g., U.S. Pat. No. 8,030,458; which is incorporated herein by reference in its entirety) and clone 1E2 (see, e.g., U.S. Pat. No. 5,532,137; which is incorporated herein by reference in its entirety). Antibodies that bind to tacrolimus are commercially available, e.g., from Fitzgerald Industries International, Hytest, Meridian Bioscience, Origene, Creative Diagnostics, and Creative BioLabs.

In some examples, the detection agent and/or capture agent can be coupled to a support, e.g., magnetic beads, colloidal particles, nanoparticles (e.g., colloidal gold or nanoparticles comprising silica, latex, polystyrene, polycarbonate, or PDVF), latex particles, or gelatin.

### (b) Hybridization Initiators and Nucleic Acid Probes

The ELFA methods and devices disclosed herein involve signal amplification via a hybridization chain reaction (HCR) in which nucleic acid probes uncoil from their hairpin structure and hybridize into a nicked double helix when triggered by a hybridization initiator strand. Each of the nucleic acid probes is conjugated to at least one detectable label, and therefore hybridization of the nucleic acid probes into the nicked double helix concentrates the detectable labels on the nucleic acid probes and dramatically enhances the detectable signal.

The HCR is initiated by a single hybridization initiator strand that binds to the sticky end of a nucleic acid probe and displaces one arm to open the hairpin. This frees the bases that were assembled into the hairpin and allows them to perform a similar displacement reaction on another nucleic acid probe. The process can continue until the nucleic acid probes are exhausted.

In some examples, the ELFA methods and devices disclosed herein involve three nucleic acid probes. In such instances, the hybridization initiator hybridizes to the first nucleic acid probe and opens the hairpin of the first nucleic acid probe to expose a binding site for the second nucleic acid probe. Binding of the second nucleic acid probe to the first nucleic acid probe opens the hairpin of the second nucleic acid probe to expose a binding site for the third nucleic acid probe. The third nucleic acid probe binds to the second nucleic acid probe, which exposes a binding site for another second nucleic acid probe, thereby allowing hybridization between the second and third nucleic acid probes to continue until their supply is exhausted.

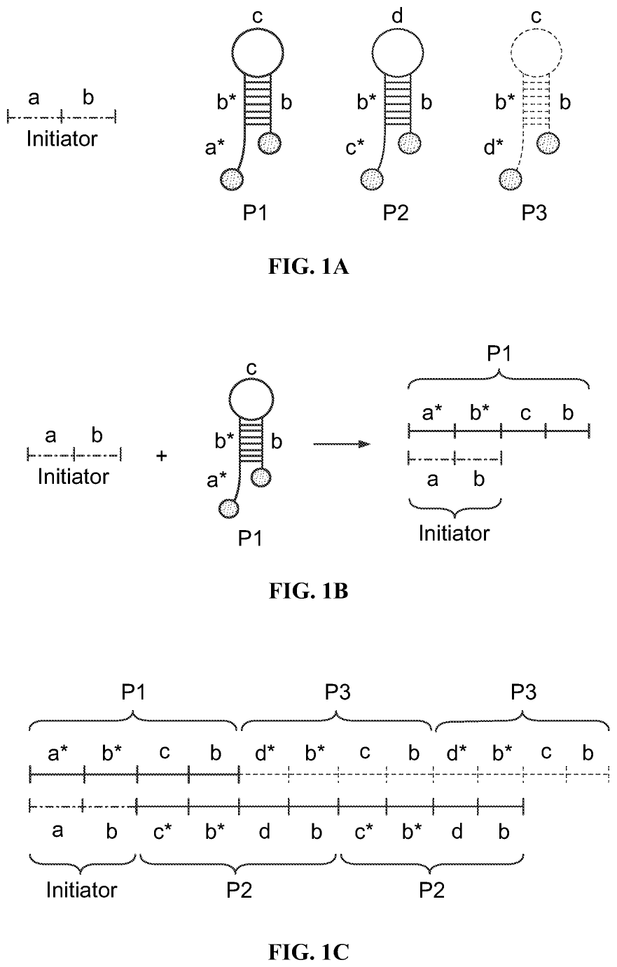

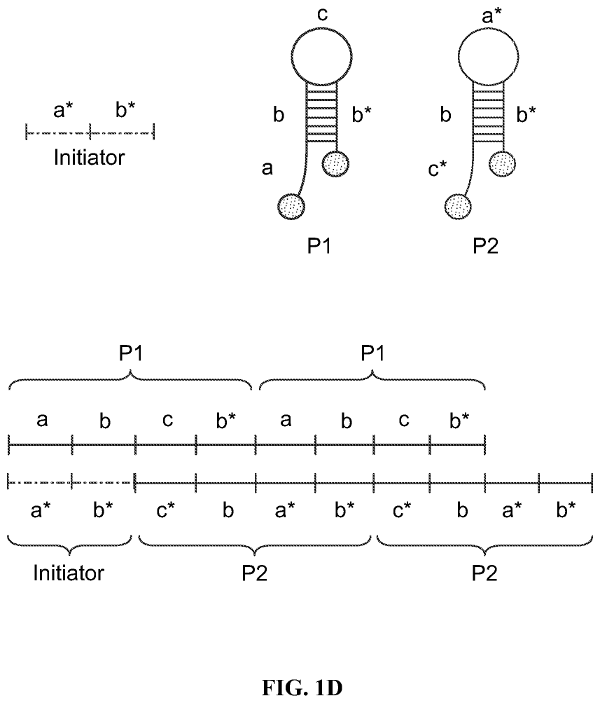

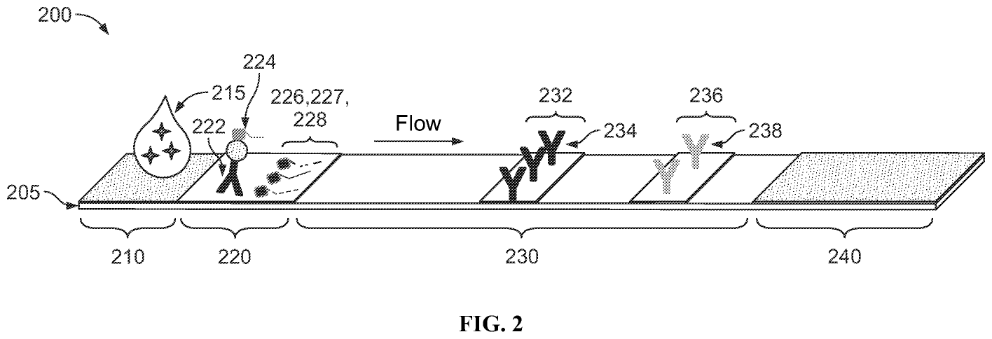

An example HCR system including a hybridization initiator and three nucleic acid probes is shown in FIG. 1A. Each of the nucleic acid probes comprise a sticky end (a*, c*, and d*, respectively), a first complementary region (b*), a loop (c and d, respectively), and a second complementary region (b). The first complementary region hybridizes to the second complementary region to form the duplex region of the hairpin.

The first nucleic acid probe comprises a sticky end (a*) and a first complementary region (b*), each of which are complementary to a portion of the hybridization initiator (a and b). As shown in FIG. 1B, hybridization of the initiator (a) to the sticky end (a*) of the first nucleic acid probe opens the hairpin and allows binding of the first complementary region (b*) to the second portion (b) of the initiator strand. Opening of the hairpin also allows the loop (c) and the second complementary region (b) to hybridize with the sticky end (c*) and the first complementary region (b*) of the second nucleic acid probe. This allows a chain reaction of hybridization events between alternating second and third nucleic acid probes to occur, resulting in formation of a nicked double helix as shown in FIG. 1C.

The first and second complementary regions (b and b*) in the first, second, and third nucleic acid probes are substantially similar so that the first complementary region (b) in one nucleic acid probe can hybridize to the second complementary region (b*) of another nucleic acid probe.

The hybridization initiator and the nucleic acid probes can be designed using any configuration that maintains the complementarity between the hybridization initiator and the first nucleic acid probe as well as the complementarity among the first, second, and third nucleic acid probes, e.g., the complementarity shown in FIG. 1A. Any sequence that maintains the complementarity can be used as a hybridization initiator and a nucleic acid probe.

The ELFA methods and devices described herein can include various HCR systems including those known in the art or described herein. In some examples, HCR systems for use in ELFA methods and devices described herein comprise one or more hybridization initiators (e.g., 1 hybridization initiator, 2 hybridization initiators, 3 hybridization initiators, or more) and two or more nucleic acid probes (e.g., 2 nucleic acid probes, 3 nucleic acid probes, 4 nucleic acid probes, 5 nucleic acid probes, or more).

For example, the ELFA methods and devices described herein can utilize HCR systems involving one hybridization initiator and two nucleic acid probes, for example, the HCR system described in Dirks and Pierce, Triggered Amplification by Hybridization Chain Reaction, PNAS (2004) vol. 101, no. 43, page 15275-15278, which is incorporated herein by reference herein in its entirety. An example HCR system including a hybridization initiator and two nucleic acid probes is shown in FIG. 1D.

The hybridization initiator and the nucleic acid probe can be various lengths. In some examples, the hybridization initiator comprises about 10 to about 100 nucleotides, e.g., about 20 to about 100 nucleotides, 30 to about 100 nucleotides, 40 to about 100 nucleotides, 50 to about 100 nucleotides, 60 to about 100 nucleotides, 70 to about 100 nucleotides, 80 to about 100 nucleotides, 90 to about 100 nucleotides, 10 to about 90 nucleotides, 10 to about 80 nucleotides, 10 to about 70 nucleotides, 10 to about 60 nucleotides, 10 to about 50 nucleotides, 10 to about 40 nucleotides, 10 to about 30 nucleotides, or 10 to about 20 nucleotides.

In some examples, the nucleic acid probe comprises about 10 to about 100 nucleotides, e.g., about 20 to about 100 nucleotides, 30 to about 100 nucleotides, 40 to about 100 nucleotides, 50 to about 100 nucleotides, 60 to about 100 nucleotides, 70 to about 100 nucleotides, 80 to about 100 nucleotides, 90 to about 100 nucleotides, 10 to about 90 nucleotides, 10 to about 80 nucleotides, 10 to about 70 nucleotides, 10 to about 60 nucleotides, 10 to about 50 nucleotides, 10 to about 40 nucleotides, 10 to about 30 nucleotides, or 10 to about 20 nucleotides.

Non-limiting examples of various hybridization initiators and nucleic acid probes for use in the ELFA devices and methods described herein are provided in Table 1.

TABLE 1
Exemplary hybridization initiators and nucleic
acid probes

| SEQ | | |
|---|---|---|
| ID | | |
| NO | Bases | Sequence |
| 1 | 48 | atatccctcgccgaatcctagactcaaagtagtctaggat |
| | | tcggcgag |
| 2 | 48 | agtctaggattcggcgagggatatctcgccgaatcctaga |
| | | ctactttg |
| 3 | 47 | agtctaggattcggcgagggatattttttctcttggaaag |
| | | aaagtgc |
| 4 | 14 | tttttgcactttct |
| 5 | 29 | agtctaggattcggcgagggatatttttt |
| 6 | 14 | tttttttccaagag |
| 7 | 14 | tttttgcactttct |
| 8 | 29 | agtctaggattcggcgagggatatttttt |

The hybridization initiator and/or nucleic acid probes for use in ELFA methods and devices described herein can be labeled with one or more detectable labels. For example, the hybridization initiator and/or the nucleic acid probe can include a detectable label at the 3′ end, the 5′ end, or both the 3′ and 5′ ends. Alternatively, or in addition to, the hybridization initiator and/or the nucleic acid probe can include a detectable label at an internal site within the hybridization initiator and/or the nucleic acid probe. The hybridization initiator and/or the nucleic acid probe can be labeled directly with a detectable label or indirectly, for example, indirectly labeled via a linker. The hybridization initiator and the nucleic acid probes can be labeled using any method known in the art.

In some embodiments, the nucleic acid probe can be labeled with a fluorophore and quencher pair to create a self-quenching nucleic acid probe. In such instances, the nucleic acid probe is labeled such that the quencher reduces fluorescence while the nucleic acid probe is in the hairpin form but not when the nucleic acid probe is incorporated into the linear HCR polymer.

The hybridization initiator and the nucleic acid probes can be DNA, RNA, or both. The hybridization initiator and the nucleic acid probes can include modified nucleic acids, e.g., nucleic acids modified at the base and/or backbone.

In some examples, the hybridization initiator and/or nucleic acid probes can be coupled to a support, e.g., magnetic beads, colloidal particles, nanoparticles (e.g., colloidal gold or nanoparticles comprising silica, latex, polystyrene, polycarbonate, or PDVF), latex particles, or gelatin.

### (c) Detectable Labels

The detection agent and/or the nucleic acid probes for use in the ELFA methods and devices as disclosed herein can be conjugated to a detectable label.

As used herein, a “detectable label” refers to any molecule that is capable of releasing a detectable signal, either directly or indirectly. Non-limiting examples of detectable labels include fluorophores, chemiluminescent compounds, radioisotopes, and colored particles (e.g., gold or silver nanoparticles).

As used herein, the term “fluorophore” (also referred to as “fluorescent label” or “fluorescent dye”) refers to a moiety that absorbs light energy at a defined excitation wavelength and emits light energy at a different wavelength. Non-limiting examples of fluorophores include Alexa Fluor® florescent dyes (e.g., Alexa Fluor® 488, Alexa Fluor® 532, Alexa Fluor® 546, Alexa Fluor® 568, Alexa Fluor® 594, Alexa Fluor® 610, Alexa Fluor® 647), cyanine dyes (e.g., Cy2®, Cy3®, Cy5®, Cy7®), fluorescent proteins (e.g., green fluorescent protein (GFP), cyan fluorescent protein (CFP), red fluorescent protein (RFP), R-Phycoerythrin (R-PE), allophycocyanin (APC)), fluorescein, BODIPY, IEDANS, EDANS, and lanthanide metals (e.g., europium, terbium, samarium).

In some embodiments, the detectable label can include a fluorophore and a quencher pair. As used herein, a “quencher” refers to a non-fluorescent molecule that can accept energy from an excited fluorophore, thereby reducing the fluorescence signal of the fluorophore. Non-limiting examples of a fluorescence quencher include Dabcyl, Tamra, and Black Hole Quenchers.

### (d) Sample Buffers

Also provided herein are sample buffers for use in the ELFA methods and devices described herein. Prior to contacting a blood sample with the ELFA device, the blood sample can be combine with a sample buffer. Without wishing to be bound by theory, combining the sample buffer with the blood sample releases a target analyte from cells without denaturing the target analyte, which maintains the structure of the target analyte and its recognition by the capture and detection agents.

Sample buffers provided herein, in some embodiments, comprise a surfactant; a salt; a sugar; and a protein. In some examples, the buffer comprises a surfactant comprising polysorbate 20, polyethylene glycol having an average molecular weight of 20,000 daltons (PEG 20), and 4-(1, 1,3,3-tetramethylbutyl)phenyl-polyethylene glycol; a sodium salt comprising sodium chloride; a sugar comprising trehalose and sucrose; and a protein comprising casein.

Non-limiting examples of a surfactant for use in sample buffers described herein include sodium dodecyl sulfate, polysorbate, octylphenol ethoxylate, 2-[4-(2,4,4-trimethylpentan-2-yl)phenoxy]ethanol, sodium deoxycholate, 3-[N,N-Dimethyl(3-myristoylaminopropyl)ammonio]propanesulfonate, polyoxyethylene lauryl ether, polyethylene glycol, 4-(1,1,3,3-tetramethylbutyl)phenyl-polyethylene glycol, t-octylphenoxypolyethoxyethanol, or a combination thereof.

Non-limiting examples of a salt for use in sample buffers described herein include a sodium salt, a potassium salt, a phosphate salt, a zinc salt, or a combination thereof. Non-limiting examples of a sodium salt for use in ELFA methods and device described herein include sodium acetate, sodium carbonate, sodium bicarbonate, sodium chloride, sodium citrate, sodium phosphate monobasic, sodium phosphate dibasic, sodium sulfate, sodium thiosulfate, sodium triphosphate, or a combination thereof.

Non-limiting examples of a sugar for use in sample buffers described herein include trehalose, sucrose, glucose, or a combination thereof.

Non-limiting examples of surfactants for use in sample buffers described herein include gelatin, albumin, casein, or a combination thereof.

In some examples, the sample buffer comprises polysorbate 20 in a total amount of about 0.05 wt % to about 10 wt % of the buffer; 4-(1,1,3,3-tetramethylbutyl)phenyl-polyethylene glycol in a total amount of about 0.001 wt % to about 4 wt % of the buffer; sodium chloride at a concentration of about 300 mM to about 1500 mM; PEG20 in a total amount of about 0.05 wt % to 10 wt % of the buffer; trehalose in a total amount of about 1 wt % to 30 wt % of the buffer; sucrose in a total amount of about 1 wt % to 30 wt % of the buffer; and casein in a total amount of about 0.1 wt % to 10 wt % of the buffer.

In some examples, the sample buffer comprises polysorbate 20 in a total amount of about 0.05 wt % to about 2 wt % of the buffer; 4-(1,1,3,3-tetramethylbutyl)phenyl-polyethylene glycol in a total amount of about 0.05 wt % to about 1 wt % of the buffer; sodium chloride at a concentration of about 2 mM to about 500 mM; PEG20 in a total amount of about 0.05 wt % to 1 wt % of the buffer; trehalose in a total amount of about 1 wt % to 5 wt % of the buffer; sucrose in a total amount of about 1 wt % to 5 wt % of the buffer; and casein in a total amount of about 0.1 wt % to 1 wt % of the buffer.

In some examples, the sample buffer comprises one or more additional components. Non-limiting examples of additional components for use in sample buffers described herein include polyoxyethylene (23) lauryl ether, potassium dihydrogen phosphate, potassium chloride, trisodium citrate, sodium dihydrogen phosphate, sodium deoxycholate, sodium dodecyl sulfate, t-octylphenoxypolyethoxyethanol, an organic solvent, or a combination thereof.

Non-limiting examples of organic solvents for use in sample buffers described herein include methanol, acetone, acetonitrile, isopropanol, dichloromethane, ethanol, or a combination thereof.

In some examples, the sample buffer comprises polyoxyethylene (23) lauryl ether. In some examples, the sample buffer comprises polyoxyethylene (23) lauryl ether in a total amount of about 0.001 wt % to about 5 wt % of the buffer. In some examples, the sample buffer comprises polyoxyethylene (23) lauryl ether in a total amount of about 0.5 wt % to about 2 wt % of the buffer.

In some examples, the sample buffer comprises potassium dihydrogen phosphate. In some examples, the sample buffer comprises potassium dihydrogen phosphate at a concentration of about 1 mM to about 500 mM. In some examples, the sample buffer comprises potassium dihydrogen phosphate at a concentration of about 1 mM to about 5 mM.

In some examples, the sample buffer comprises potassium chloride. In some examples, the sample buffer comprises potassium chloride at a concentration of about 1 mM to about 200 mM. In some examples, the sample buffer comprises potassium chloride at a concentration of about 3 mM to about 30 mM.

In some examples, the sample buffer comprises trisodium citrate. In some examples, the sample buffer comprises trisodium citrate at a concentration of about 5 mM to about 500 mM. In some examples, the sample buffer comprises trisodium citrate at a concentration of about 10 mM to about 100 mM.

In some examples, the sample buffer comprises sodium dihydrogen phosphate. In some examples, the sample buffer comprises sodium dihydrogen phosphate at a concentration of about 1 mM to about 100 mM. In some examples, the sample buffer comprises sodium dihydrogen phosphate at a concentration of about 5 mM to about 10 mM.

In some examples, the sample buffer comprises sodium deoxycholate. In some examples, the sample buffer comprises sodium deoxycholate in a total amount of about 0.02 wt % to about 15 wt % of the buffer. In some examples, the sample buffer comprises sodium deoxycholate in a total amount of about 0.5 wt % to about 1 wt % of the buffer.

In some examples, the sample buffer comprises sodium dodecyl sulfate. In some examples, the sample buffer comprises sodium dodecyl sulfate in a total amount of about 0.5 wt % to about 5 wt % of the buffer. In some examples, the sample buffer comprises sodium dodecyl sulfate is present in a total amount of about 0.5 wt % to about 2 wt % of the buffer.

In some examples, the sample buffer comprises t-octylphenoxypolyethoxyethanol. In some examples, the sample buffer comprises t-octylphenoxypolyethoxyethanol in a total amount of about 0.001 wt % to about 4 wt % of the buffer. In some examples, the sample buffer comprises t-octylphenoxypolyethoxyethanol in a total amount of about 0.5 wt % to about 2 wt % of the buffer.

In some examples, the sample buffer comprises an organic solvent. In some examples, the sample buffer comprises acetonitrile. In some examples, the sample buffer comprises acetonitrile in a total amount of about 1 wt % to about 50 wt % of the buffer. In some examples, the sample buffer comprises acetonitrile in a total amount of about 5 wt % to about 30 wt % of the buffer.

The sample buffer can comprise one or more components for detection of a target analyte. For example, the sample buffer comprises a detection agent, a hybridization initiator, one or more nucleic acid probes, or a combination thereof. In some examples, the detection agent is present in the sample buffer at a concentration in a range of 5 nM to 40 nM. In some examples, the hybridization initiator is present in the sample buffer at a concentration in a range of 5 nM to 40 nM. In some examples, each nucleic acid probe is present in the sample buffer at a concentration in a range of 5 nM to 40 nM.

### II. Enhanced Lateral Flow Assay (ELFA) Devices

Provided herein are ELFA devices for measuring an analyte in a sample. Reference is now made to FIGS. 2-4, which illustrate pictorially various embodiments of exemplary ELFA devices described herein.

As shown in FIG. 2, the ELFA device 200, in some embodiments, comprises a sample region 210 that provides absorption of a sample 215; a conjugate region 220 comprising a detection agent 222, a hybridization initiator 224, and nucleic acid probes 226, 227, and 228; a detection region 230 comprising a test zone 232 comprising a capture agent 234 and a control zone 236 comprising a control agent 238; and an absorbent region 240 that provides absorption of excess reagents and maintains a lateral flow along the detection region. As also shown in FIG. 2, the sample region 210, the conjugate region 220, the detection region 230, and the absorbent region 240 can be provided on (e.g., disposed on, coupled to, or mounted on) a substrate 205.

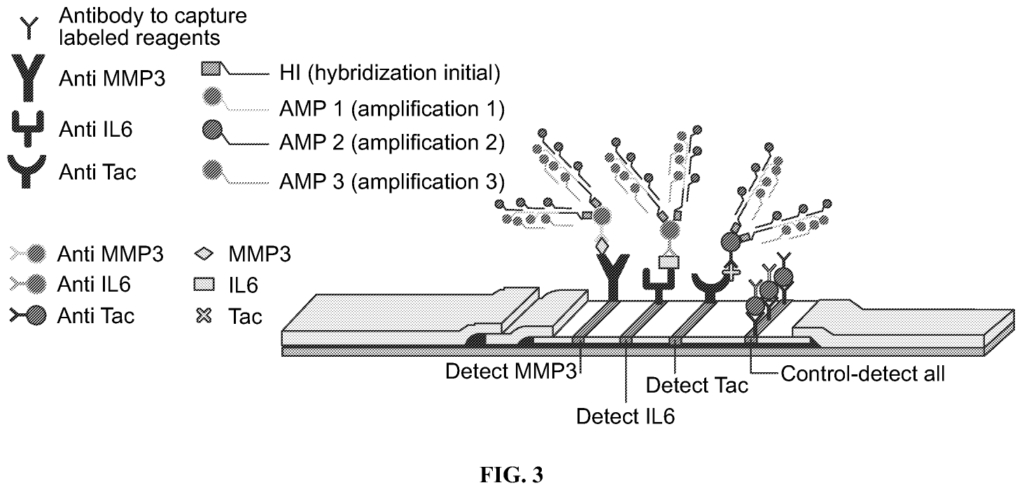

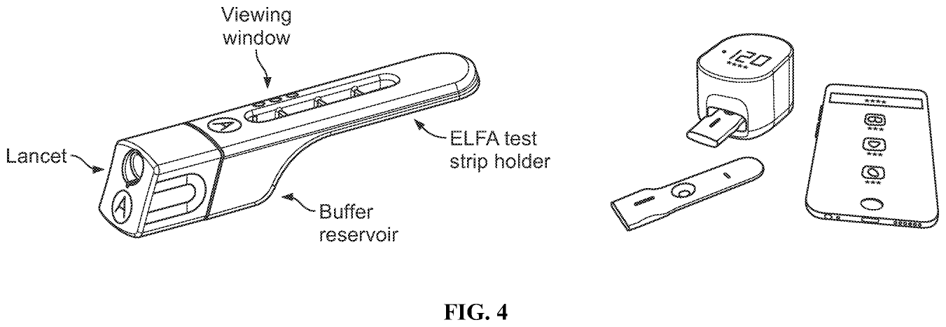

As shown in FIG. 3, ELFA devices can comprise one or more additional test zones for detecting a second analyte in a sample. For example, as shown in FIG. 3, the ELFA device includes two additional test zones for detection of inflammation markers MMP3 and IL6, respectively. The additional test zones include capture agents that bind to MMP3 or IL6 in the sample. MMP3 and IL6 in the test zones can then be detected using detection agents that binds MMP and IL6 and any of the HCR systems described herein.

As shown in FIG. 4, ELFA devices can comprise a housing, which can be configured to provide a user with a fingerstick device for collecting a blood sample and a sample buffer for combining with the sample prior to contacting the blood sample with the ELFA device. In some embodiments, the housing can comprise one or more openings such as a sample port for contacting the sample with the device and a viewing window for viewing the test and control zones. The housing or a portion of the housing can be removable. As also shown in FIG. 4, the ELFA device can be inserted into a signal detection device to obtain quantified results, which can be displayed on a mobile device such as a mobile phone. Depending on the signal to be detected from the detectable label, the signal detection device can include a colorimetric reader, a fluorescent reader, or both.

The detection agent and HCR system components can be immobilized on the conjugate region using any method known in the art. The detection agent and HCR system components can immobilized to a surface of the conjugate region, directly or indirectly. In some embodiments, the detection agent is immobilized to a surface via a covalent bond or a non-covalent bond. In some embodiments, the detection agent is immobilized to a surface via a linker. Non-limiting examples of linkers for immobilizing the detection agent to a surface include carbon-containing chains, polyethylene glycol, nucleic acids, monosaccharides, biotin, avidin, and peptides.

As used herein, the term “immobilized” refers to reversibly and irreversibly immobilized molecules (e.g., detection agents, capture agents, hybridization initiators, nucleic acid probes). Reversibly immobilized molecules are immobilized in a manner that allows the molecules, or a portion thereof (e.g., at least 25%, 50%, 60%, 75%, 80% or more of the molecules), to be removed from their immobilized region without substantial denaturation or aggregation. In some examples, a molecule can be reversibly immobilized on a region by contacting a solution containing the molecule with the region, thereby soaking up the solution, and then drying the solution containing the molecule. The reversibly immobilized molecule can then be removed by contacting the region with the sample, thereby solubilizing the reversibly immobilized molecule.

Any concentration of detection agent suitable for capturing a target analyte and allowing production of a detectable signal can be applied on the conjugate region. In some examples, a concentration of the detection agent applied on the conjugate region is in a range of 5 nM to 40 nM, e.g., 10 nM to 40 nM, 15 nM to 40 nM, 20 nM to 40 nM, 25 nM to 40 nM, 30 nM to 40 nM, 35 nM to 40 nM, 5 nM to 35 nM, 5 nM to 30 nM, 5 nM to 25 nM, 5 nM to 20 nM, 5 nM to 15 nM, or 5 nM to 10 nM.

The capture agent can be immobilized on the test zone of the detection region using any method known in the art. The capture agent can be immobilized on, or bound to, a surface of the detection region, directly or indirectly. In some embodiments, the capture agent is immobilized to a surface via a covalent bond or a non-covalent bond. In some embodiments, the capture agent is immobilized to a surface via a linker. Non-limiting examples of linkers for immobilizing the capture agent to a surface include carbon-containing chains, polyethylene glycol, nucleic acids, monosaccharides, biotin, avidin, and peptides.

Any concentration of capture agent suitable for capturing a target analyte and allowing production of a detectable signal can be applied on the detection region. In some examples, a concentration of the capture agent applied on the detection region is in a range of 5 nM to 40 nM, e.g., 10 nM to 40 nM, 15 nM to 40 nM, 20 nM to 40 nM, 25 nM to 40 nM, 30 nM to 40 nM, 35 nM to 40 nM, 5 nM to 35 nM, 5 nM to 30 nM, 5 nM to 25 nM, 5 nM to 20 nM, 5 nM to 15 nM, or 5 nM to 10 nM.

It should be appreciated that various embodiments of the ELFA device, including various components in the ELFA device described herein, can be formed with any materials suitable for performing a lateral flow assay. For example, the ELFA device can include any sample region suitable for absorbing a sample; any conjugate region suitable for depositing detection agents and HCR components; any detection region for immobilizing capture agents; and any detection and capture agents suitable for binding an analyte.

Any detection region suitable for immobilizing capture agents and supporting lateral flow of a sample can be used in ELFA devices described herein. In some examples, the detection region comprises a membrane. Non-limiting examples of a membrane include a nitrocellulose membrane, a nylon membrane, a cellulose membrane, a polyvinylidine fluoride membrane, a polycarbonate membrane, a polypropylene membrane, a polyethylene membrane, a polytetrafluoroethylene membrane, and a poly-paraphenylen terephthalamide membrane.

Any sample region suitable for absorbing a sample and maintaining a lateral flow of the sample along the substrate can be used in ELFA devices described herein. In some embodiments, the sample region comprises cellulose or glass fiber.

Any conjugate region suitable for depositing reagents and maintaining a lateral flow of a sample along the substrate can be used in ELFA devices described herein. In some embodiments, the conjugate region comprises cellulose or class fiber.

Any absorbent region suitable for absorbing excess reagents and maintaining a lateral flow of a sample along the substrate can be used in ELFA devices described herein. In some embodiments, the absorbent region comprises cellulose or class fiber.

Any substrate on which one or more regions can be provided can be used in ELFA devices described herein. In some embodiments, the substrate comprises plastic (e.g., styrene, polycarbonate, polypropylene, polyethylene, polyvinyl chloride).

In some examples, the ELFA device includes a conjugate region without the detection agent and the HCR system components or the ELFA device includes no conjugate region. In such instances, the detection agent and the HCR system components can be combine with the sample prior to applying the sample to the ELFA device. For example, the detection agent and the HCR system components can be included in the sample buffer, which is combined with the sample and contacted to the ELFA device. Alternatively, or in addition to, the detection agent and the HCR system components can be included in the sample region and/or the detection region.

### III. Enhanced Lateral Flow Assay (ELFA) Methods

Also provided herein are methods for detecting an analyte in a sample. The assay methods disclosed herein involve the use of a detection agent, a capture agent, a hybridization initiator, and a plurality of nucleic acid probes, which are all disclosed herein. In some examples, the detection agent, the hybridization initiator, and each nucleic acid probe is labeled with at least one detectable label.

To perform the assay method disclosed herein, a sample suspected of containing an analyte is brought in contact with the ELFA device under conditions allowing for formation of a complex comprising the detection agent, the analyte, and the capture agent. A presence or a level of the analyte in the sample can be detected by measuring a signal released from the detectable labels, which can be conjugated to the detection agent, the hybridization initiator, and the nucleic acid probes.

As used herein, the term “contacts” refers to an exposure of a sample with an ELFA device described herein for a period sufficient for the formation of a complex comprising the hybridization initiator, the nucleic acid probes, the detection agent, the analyte, and the capture agent, if any.

In some examples, ELFA methods are performed using a device configured as shown in FIG. 2. In such instances, a sample 215 can be applied to the sample region 210 of the ELFA device 200. The sample moves along the sample region 210 to the conjugate region 220 via capillary action. When the sample reaches the conjugate region 220, the sample solubilizes the detection agent 222 labeled with the hybridization initiator 224 and solubilizes the nucleic acid probes 226, 227, and 228. The detection agent can then bind to the analyte to form a detection agent-analyte complex. Also, the hybridization initiator can trigger the HCR to form a nicked double helix comprising the nucleic acid probes, which is attached to the detection agent. When the sample reaches the test zone 232 in detection region 230, the detection agent-analyte complex binds to the capture agent 234 to form a “sandwich”. In this example, the sandwich therefore comprises the capture agent bound to analyte, which is bound to the detection agent, which is labeled with the nucleic acid probes via the hybridization initiator. After moving into the test zone 232, the sample continues to advance along the detection region 230 to the control zone 238 and into the absorbent region 240, which acts as a wick to pull the sample away from the detection region 230, thus removing any excess material from the detection region 230.

Methods described herein encompass combining the sample with a sample buffer prior to contacting the sample with the device. In such instances, the sample is incubated with the sample buffer for a time sufficient to facilitate binding of an analyte in the sample to a detection agent and a capture agent on the ELFA device. For example, when the sample comprises cells, the sample can be incubated with a sample buffer for a time sufficient to release an analyte from the cells.

In some embodiments, prior to contacting the sample with the ELFA device, the sample is incubated with the sample buffer for 1 to 15 minutes, e.g., 1 to 14 minutes, 1 to 13 minutes, 1 to 12 minutes, 1 to 11 minutes, 1 to 10 minutes, 1 to 9 minutes, 1 to 8 minutes, 1 to 7 minutes, 1 to 6 minutes, 1 to 5 minutes, 1 to 4 minutes, 1 to 3 minutes, 1 to 2 minutes, 2 to 15 minutes, 3 to 15 minutes, 4 to 15 minutes, 5 to 15 minutes, 6 to 15 minutes, 7 to 15 minutes, 8 to 15 minutes, 9 to 15 minutes, 10 to 15 minutes, 11 to 15 minutes, 12 to 15 minutes, 13 to 15 minutes, or 14 to 15 minutes.

ELFA methods described herein encompass detecting an analyte, or a lack thereof, in various samples. In some embodiments, the sample is a biological sample obtained from a subject. In some embodiments, the biological sample is a blood sample, e.g., a whole blood sample, a serum sample, or a plasma sample. When the sample is a serum sample or a plasma sample, methods described herein can comprise isolating plasma or serum from blood. In some embodiments, blood cells in a blood sample can be lysed prior to contacting the blood sample with any of the ELFA devices described herein.

Any analyte in a sample can be detected using the ELFA methods and device described herein. As used herein, the term “analyte” refers to any target molecule for detection using the ELFA methods and devices described herein. Non-limiting examples of an analyte include a drug, a protein, a nucleic acid, a polysaccharide, a lipid, an antigen, and a growth factor.

In some examples, the analyte is a drug. Non-limiting examples of a drug include an immunosuppressant, an antibiotic, an anticoagulant, an antidepressant, a bronchodilator, an anticonvulsant, an antiarrhythmic, an aminoglycoside, or a combination thereof. Non-limiting example of an immunosuppressant include tacrolimus, ascomycin, everolimus, pimecrolimus, ridaforolimus, temsirolimus, umirolimus, ascomycin, sirolimus, cyclosporine, zonotarolimus, or a combination thereof.

Methods described herein can comprise obtaining a biological sample from the subject, combining the sample with a sample buffer, and contacting the sample with any of the ELFA devices described herein. In some embodiments, methods comprise isolating plasma or serum or other blood components from whole blood, and then contacting the isolated sample with any of the ELFA devices described herein. In other embodiments, whole blood is contacted with any of the ELFA devices described herein.

Methods described herein utilize a small volume of sample, e.g., a sample obtained from a fingerstick. Such samples can be obtained by the subject. In some embodiments, the volume of the sample is 1 to 50 μL, e.g., 1 to 45 μL, I to 40 μL, 1 to 35 μL, 1 to 30 μL, 1 to 25 μL, 1 to 20 μL, 1 to 15 μL, I to 10 μL, 1 to 5 μL, 5 to 50 μL, 10 to 50 μL, 15 to 50 μL, 20 to 50 μL, 25 to 50 μL, 30 to 50 μL, 35 to 50 μL, 40 to 50 μL, or 45 to 50 μL.

The term “subject” or “patient” can be used interchangeably and refers to a subject who needs the analysis as described herein. In some embodiments, the subject is a human or a non-human mammal (e.g., cat, dog, horse, cow, goat, or sheep).

In some embodiments, the subject has received an organ or a tissue transplant and is undergoing treatment with an immunosuppressant. In such instances, the subject may have received a transplant of any organ or any tissue (e.g., kidney, heart, liver, intestine, thymus, pancreas, lung, skin, bone, bone marrow, tendon, heart valve, cornea, nerve, or vein).

ELFA methods and devices described herein can be used to select a dose of a drug for a subject. Such methods include determining the level of a drug (e.g., an immunosuppressant such as tacrolimus) in a biological sample (e.g., a whole blood sample, a serum sample, a plasma sample) collected from a subject (e.g., a human patient who is taking the drug). The level of drug in the biological sample can then be measured using any of the ELFA methods and devices described herein. The biological sample can be collected from the subject before and after the treatment with a drug and/or during the course of the treatment.

In some examples, the ELFA methods and device described herein can be used to select a dose of a drug for a subject based on the therapeutic index of the drug. For example, if the subject is identified as having a level of drug in their system that is higher than a dose falling within the therapeutic index of the drug, a lower dose and/or a lower frequency of the dose of the drug can be administered to the identified subject. In another example, if the subject is identified as having a level of drug in their system that is lower than a dose falling within the therapeutic index of the drug, a higher dose and/or a greater frequency of the dose of the drug can be administered to the identified subject.

Accordingly, in some examples, determining a dose of drug depends on the therapeutic index of the drug. Alternatively, or in addition to, determining a dose of drug depends on whether the treatment with the drug is effective for the subject's disease or condition.

### IV. Kits for Detecting Analytes in Blood Samples

The present disclosure also provides kits for detecting analytes in blood samples from a subject. Such kits can include any of the ELFA devices and/or the sample buffers described herein. The kit can also include instructions for practicing any of the ELFA methods described herein. Instructions supplied in the kits of the present disclosure are typically written instructions on a label or package insert. The kits provided herein are in suitable packaging. Suitable packaging includes, but is not limited to, containers, bottles, vials, and flexible packaging. Kits can include additional components, for example, a component for collecting a blood sample such as a fingerstick device and/or a component for detecting a signal such as a colorimetric reader, a fluorescent reader, or both.

### EXAMPLES

In order that the invention described can be more fully understood, the following examples are set forth. The examples described in this application are offered to illustrate the methods and compositions provided herein and are not to be construed in any way as limiting their scope.

### Example 1: Development of an Enhanced Lateral Flow Assay (ELFA) for Detecting Tacrolimus (Tac)

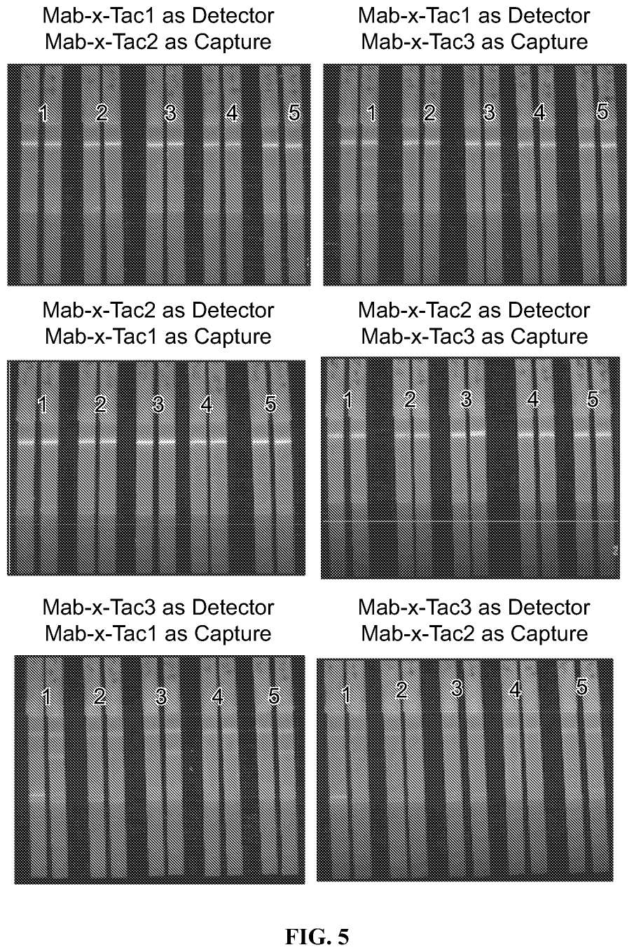

Reagents and high affinity monoclonal antibodies were screened to determine the most effective tacrolimus recognition antibodies, conjugation reagents, and immobilization substrates. ELFA substrates were fabricated for the tacrolimus assay, using a sandwich immunoassay format that enabled the assay to recognize multiple Tac-epitopes. Three antibodies (Mab-x-Tac 1, Mab-x-Tac 2, and Mab-x-Tac3) were tested in both capture (striped on the nitrocellulose membrane) and detector (conjugated to fluorescent reporter molecule) configurations, so that in the presence of Tac the test/capture lines fluoresce with an intensity proportional to the concentration detected. Five running buffers were used in each iteration. Based on the results shown in FIG. 5, Mab-x-Tac 1 (as capture) and Mab-x-Tac3 (as detector) in combination with Buffer 3 were used for further studies.

A matrix of antibody pairs tested in the LFA methods and devices described herein is shown in Table 2. The eight antibody pairs that effectively detected tacrolimus include the following capture/detector antibody pairs: Hytest (Hy)/Fitzgerald (F1), Merdian (Me)/Fitzgerald (F1), Creative Diagnostics (CD1)/Fitzgerald (F1), Creative Diagnostics (CBL)/Fitzgerald (F1), Hytest (Hy)/Creative BioLabs (CD2), Merdian (Me)/Creative BioLabs (CD2), Creative Diagnostics (CD1)/Creative Diagnostics (CD1), and Creative Diagnostics (CD1)/Creative Diagnostics (CBL). The identified antibody pairs were found to be highly specific to tacrolimus (differentiated from both other immunosuppressant drugs and Tac analogs) and highly sensitive.

TABLE 2
Results for detection of Tac using various capture:detector antibody pairs

| | Membrane | | | | | | | |
|---|---|---|---|---|---|---|---|---|
| | striped | | | | | | | |
| | capture | Latex conjugated antibodies | | | | | | |
| Company | antibodies | F1 | F2 | Hy | Or | CD1 | CBL | CD2 |
| Fitzgerald | F1 | No binding | Nonspecific | No binding | Nonspecific | No binding | No binding | Nonspecific |
| | F2 | Weak binding | No binding | No binding | Nonspecific | No binding | No binding | Nonspecific |
| Hytest | Hy | Binding | No binding | No binding | No binding | No binding | No binding | Binding |
| Meridian | Me | Binding | No binding | Nonspecific | No binding | No binding | No binding | Binding |
| Origene | Or | Nonspecific | Nonspecific | No binding | No binding | No binding | No binding | Nonspecific |
| Creative | CD1 | Binding | No binding | Nonspecific | No binding | No binding | No binding | Binding |
| Diagnostics | CD2 | Nonspecific | No binding | No binding | No binding | No binding | No binding | No binding |
| Creative | CBL | Binding | Nonspecific | No binding | Nonspecific | No binding | No binding | Binding |
| BioLabs | | | | | | | | |

Antibody sources: Fitzgerald (F1, cat. no. 20-1195; F2, cat. no. 20-1197); Hytest (Hy, cat. no. 4FK42); Meridian (Mer, cat. no. G01239M); Origene (Or; cat no. AP09835PU-N); Creative Diagnostics (CD1, cat no. DMAB4350; CD2, cat no. DPABY-986); Creative BioLabs (CBL, CBL-rAB-0516YJ).

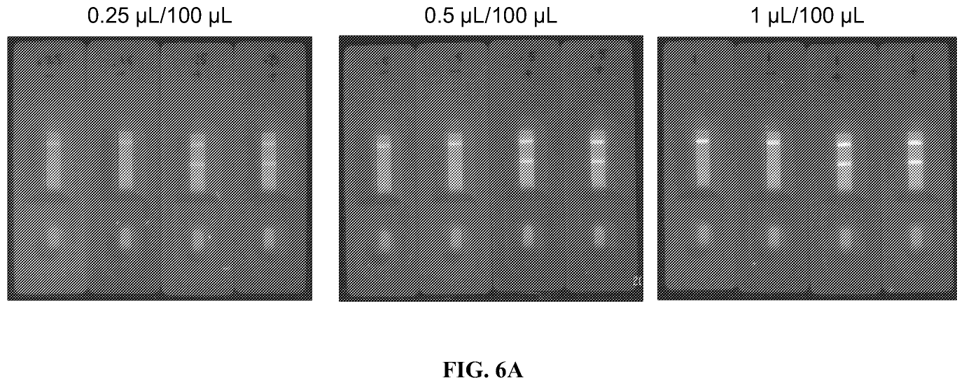

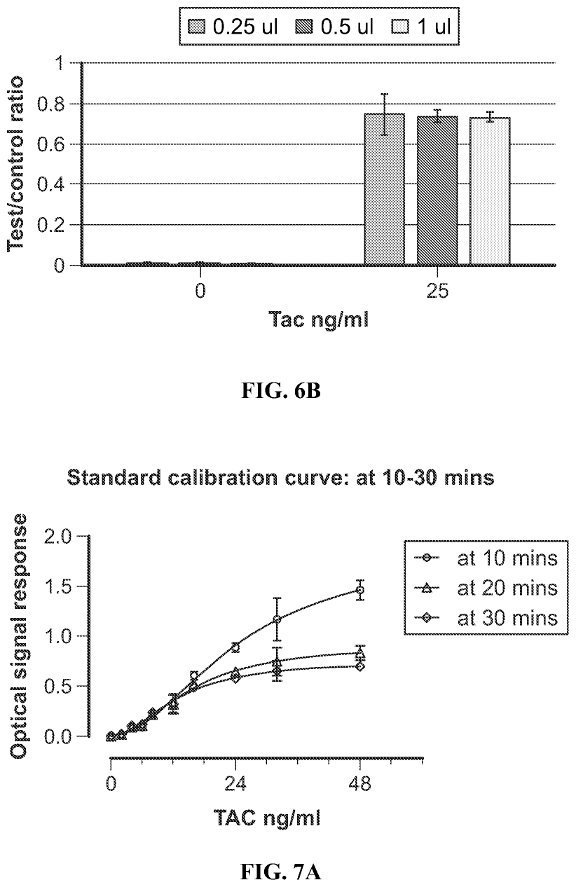

Various amounts of detector antibody were tested using samples spiked with Tac. Assays were performed with 0.25, 0.5, and I μL of conjugated detector antibody and either 0 and 25 ng/ml spiked Tac in a 100 μL sample. Photographs and plotted values of the assay results are provided in FIG. 6A and FIG. 6B, respectively. 0.5 μL of conjugated detector antibody was selected for further testing because it exhibited higher repeatability and a strong signal.

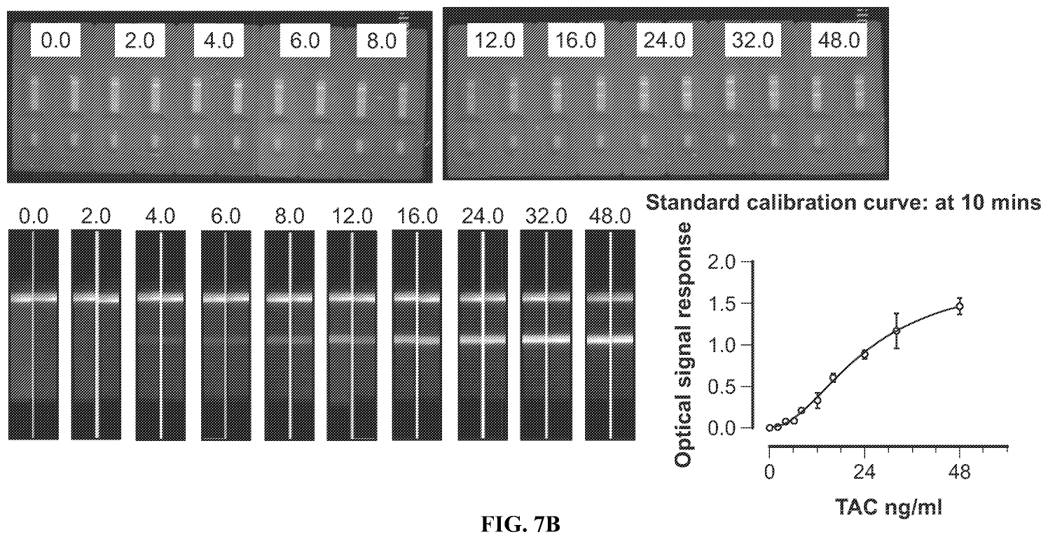

A preliminary serial dilution experiment was conducted to evaluate a standard calibration curve for level of detection (LOD) and level of saturation (LOS). Samples containing Tac at 0, 2, 4, 6, 8, 12, 16, 24, 32, and 48 ng/ml were tested. Assay strips were enclosed in a standard on-port cassette, and signal measurements were detected on a commercial available fluorescence imaging device. To evaluate the assay settings, data for the standard calibration curve (SCC) was recorded at 10, 20, and 30 minute endpoints (time from applying the sample to reading) (FIG. 7A). As seen in FIG. 7B, the 10 minute runtime resulted in the best measurement range, as 20 and 30 minute runtimes resulted in saturation at Tac levels exceeding 24 ng/ml.

Taken together, these results demonstrate that quantitative detection of tacrolimus in spiked buffer samples can be achieved using the ELFA devices and methods described herein.

### Example 2: Detection of Tac in Plasma Samples Using an ELFA

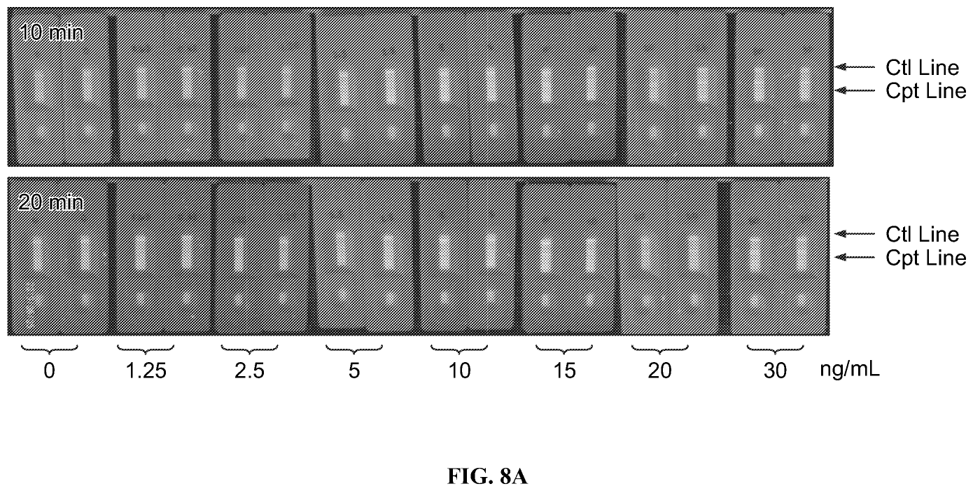

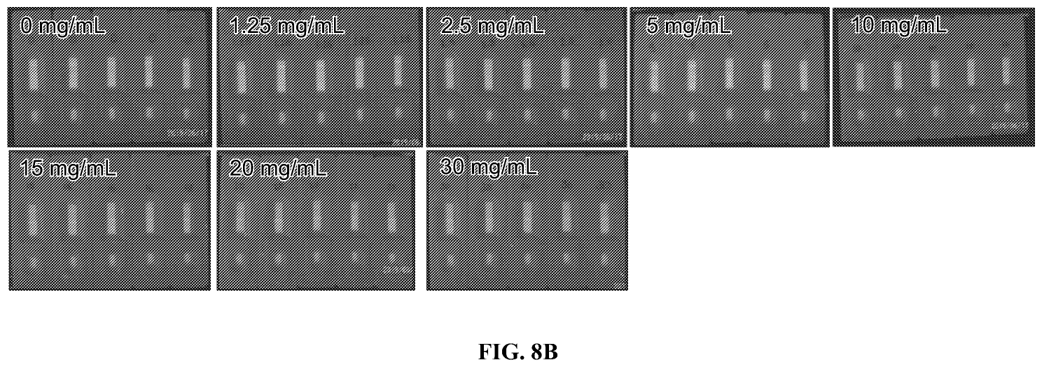

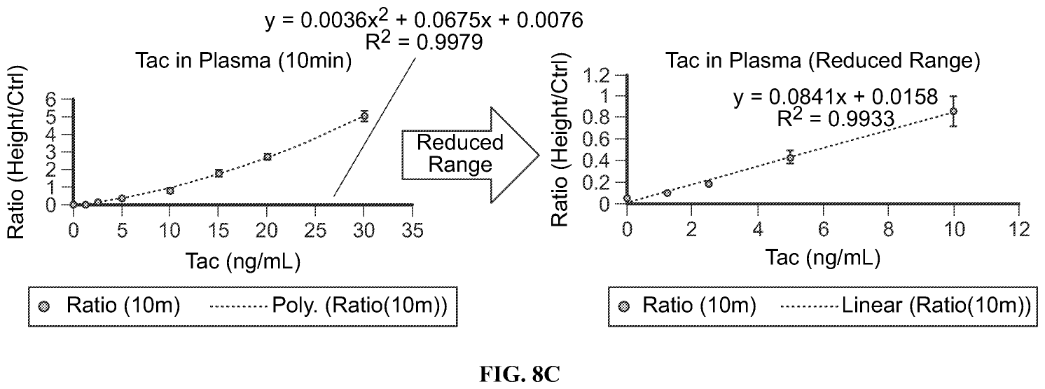

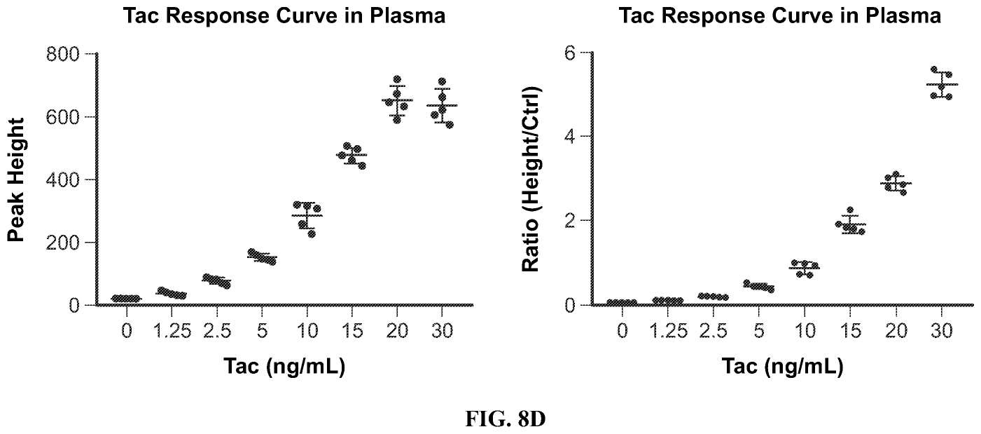

To test the assay with plasma samples, plasma samples were acquired from a commercial supplier of healthy donor blood, BioIVT. The assay was performed using Mab-x-Tac1 as the capture antibody and Mab-x-Tac3 as the detector antibody. The assay was calibrated for measurement range and assay saturation with plasma samples spiked with Tac at 0, 1.25, 2.5, 5, 10, 15, 20, and 30 ng/ml (FIGS. 8A-8B). A reduced range with 0-10 ng/ml exhibited a linear correlation with the capture/control ratio measurements (FIG. 8C).

Assay repeatability was tested with a serial dilution of Tac in plasma at n=5, comparing capture line peak height to capture/control ratio measurements (FIG. 8D). Assay saturation was observed at 20 ng/ml, which is beyond clinically relevant values, confirming that the working assay measurement range of 0-20 ng/ml will adequately address the therapeutic concentrations in blood (FIG. 8D). Based on that initial assay measurement, the level of detection was determined to be 2 ng/ml.

Taken together, these results demonstrated that quantitative detection of Tac in spiked plasma samples can be achieved using the ELFA devices and methods described herein.

### Example 3: Shelf-Life Aging Studies

Room temperature storage condition studies for the Tac-ELFA were performed to assess the assay fabrication, drying, and packaging specifications. The cassettes housing the Tac-ELFA test strips with dried capture and control antibodies, dried fluorescently conjugated detector antibody, and treated sample pads, were fabricated in a lot of 50 substrates. Tac-ELFA test strips were stored at room temperature and then stored Tac-ELFA test strips were used to detect Tac in spiked (5 ng/mL) and unspiked plasma sample over a 1-year period (assays performed on Day 1, 7, 14, 45, 75, 105, 135, 167, 195, 224, 254, 284, 314, 344, and 374).

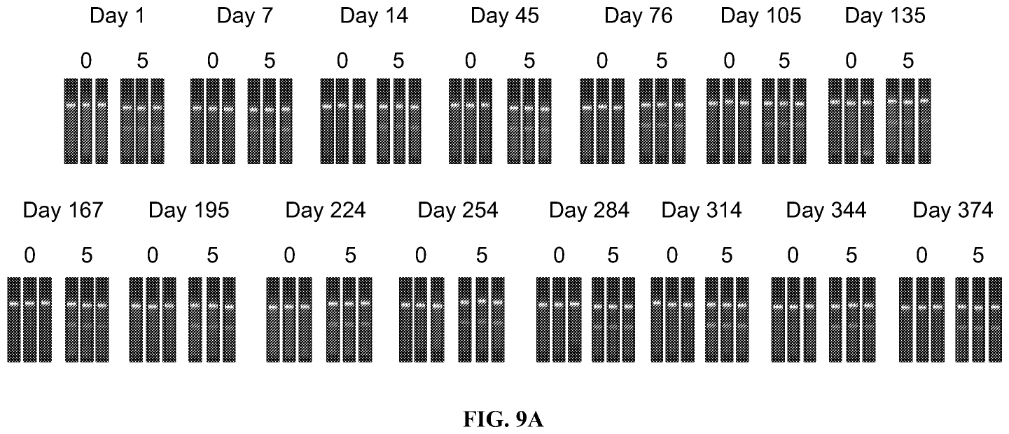

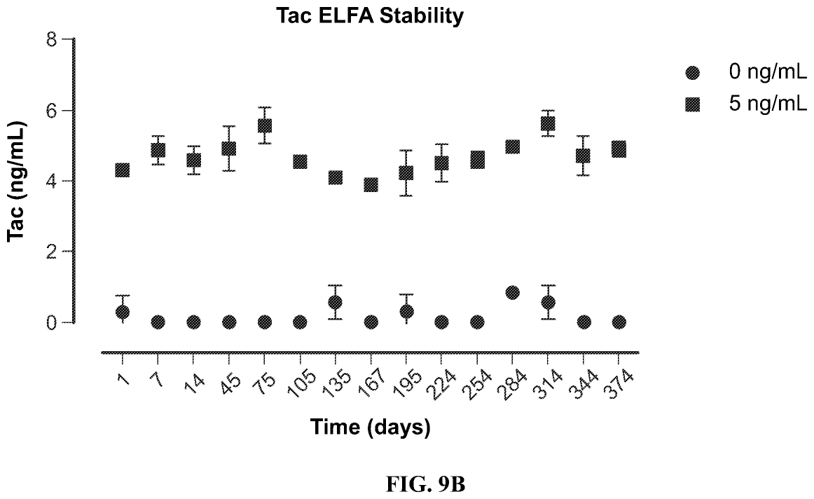

Stability was demonstrated over approximately 12 months (374 days) of storage at room (FIGS. 9A-9B). Acceptable stability was observed in 5 ng/ml spiked samples compared to negative samples. Reagent stability, conjugate flow, and lack of non-specific interaction were also observed (FIGS. 9A-9B).

Thus, these results demonstrated that room temperature storage does not compromise the performance of the ELFA devices and methods described herein.

### Example 4: ELFA Performance with Plasma Samples

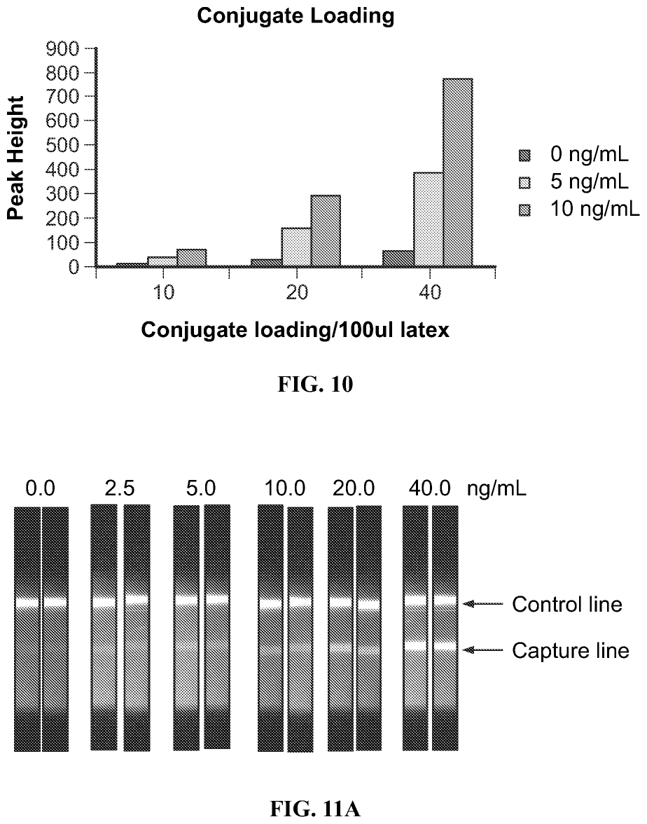

The ELFA platform was selected to evaluate performance in pooled human plasma samples acquired through the commercial biofluid supplier, BioIVT. These samples are treated with anticoagulants to avoid rapid hemolysis upon collection. The enhanced lateral flow Tac assays were tested using spiked samples with known levels of Tac. Initial testing with plasma resulted in significant aggregation at the sample pad-membrane interface, which was presumed to be caused in part by the plasma matrix effect and in part steric hindrance associated with the conjugate interacting with samples containing Tac. Three levels of conjugates were used to assess this effect (FIG. 10). Based on these results, conjugate levels of 20 μg/100 μL and 40 μg/100 μL were tested for a dose response curve. 20 μg/100 μL and 40 μg/100 μL conjugate levels showed acceptable results, with resolved sensitivity of 5 ng/ml in plasma (FIG. 10).

### Example 5: ELFA Performance with Whole Blood Samples

The ELFA platform was tested using whole blood samples spiked with tacrolimus. As shown in FIGS. 11A-11C, tacrolimus (2.5 ng/ml to 40.0 ng/mL) was detected in whole blood samples using the ELFA platform.

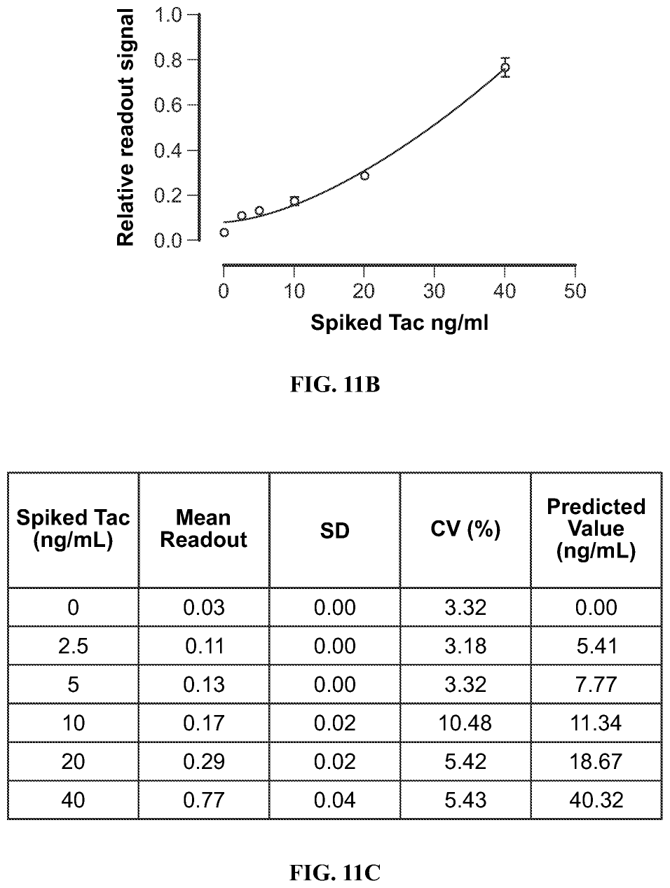

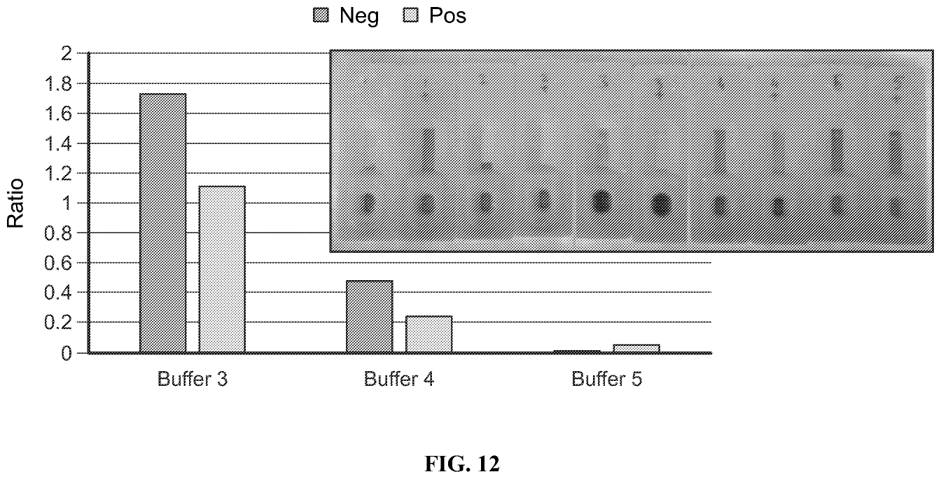

To address problems with whole blood-based assays, red-blood-cell filtration pads were integrated into the ELFA platform. Whole blood testing was performed with five buffers as diluents (FIG. 12). An inconsistent flow of whole blood was observed with all five buffers, but the buffer identified as “buffer 3” in the figure provided acceptable results based on sample flow, conjugate recognition of Tac, and efficiency of red-blood-cell removal. Based on these initial results, the buffer identified as “buffer 3” was selected for further analysis for use as the Tac assay lysis buffer.

### Example 6: ELFA Performance with Whole Blood Samples Treated with Lysis Buffer

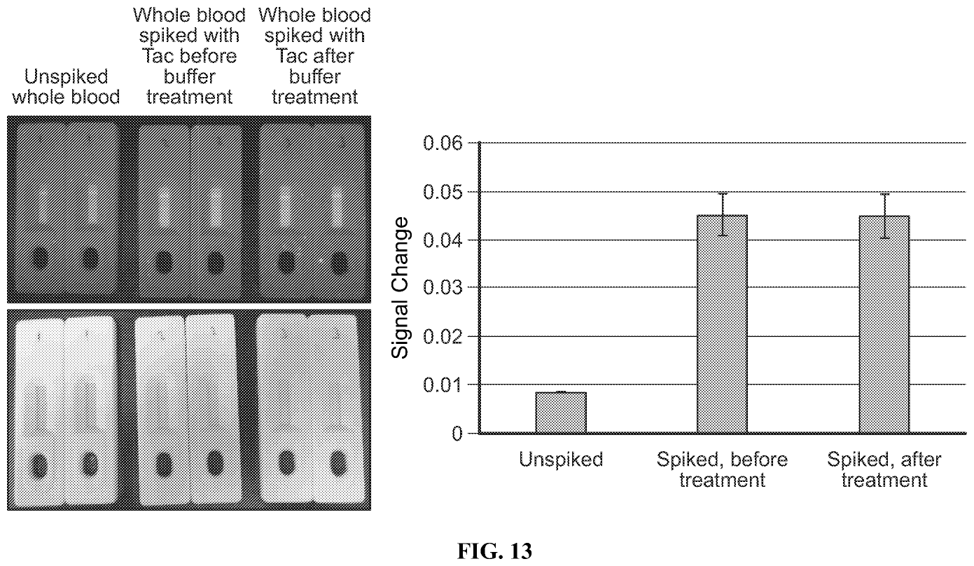

To better evaluate the effect of the lysis buffer on the ELFA platform's ability to detect Tac, we designed a test consisting of samples of unspiked whole blood, whole blood spiked with 10 ng/mL Tac before treatment with lysis buffer, and whole blood spiked with 10 ng/ml Tac after treatment with lysis buffer (FIG. 13). The resulting signal was similar to that from the same level of Tac before and after the whole blood treatment, demonstrating that treatment of whole blood with the lysis buffer does not affect efficacy of the ELFA platform's binding to Tac.

### Example 7: Cross-Reactivity in the ELFA Platform

The ELFA platform can include an immune-sandwich lateral flow format, and it was determined that one of the antibodies (Ab #1) binds to site C32 on Tac, and the other (Ab #2) to site C22 on Tac. This enables the ELFA platform to not cross-react with the Tac metabolites and analogs listed in Table 3.

TABLE 3
Tac metabolites and analogs

| Metabolite/Analog Name | Metabolite/Analog Name |
|---|---|
| Tacrolimus | 13,31-O-didesmethyl |
| | tacrolimus (MVI) |
| 13-O-desmethyl tacrolimus (MI) | 13,15-O-dedesmethyl |
| | tacrolimus (MVII) |
| 31-O-desmethyl tacrolimus (MII) | MVIII |
| 15-O-desmethyl tacrolimus (MIII) | C22 oxime tacrolimus |
| 12-O-hydroxyl tacrolimus (MIV) | C24 succinate tacrolimus |
| 15,31-O-didesmethyl tacrolimus (MV) | C32 succinate tacrolimus |

Tac-ELFA cross-reactivity was evaluated against two other common immunosuppressant drugs (ISDs), cyclosporine and sirolimus. Antibody cross-reactivity to these ISDs was measured with the Tac-ELFA at two reference levels (5 and 20 ng/ml) of tacrolimus in whole blood, covering the target detection range, with six replicates of each test. Cyclosporine and sirolimus were spiked to reference whole blood samples at the highest therapeutic levels (25 and 300 ng/mL, respectively) with added tacrolimus, while the test samples contained only tacrolimus. Cross-reactivity was calculated as the mean excess tacrolimus concentration detected in the reference samples, compared to the test samples (Table 4 and Table 5).

TABLE 4
Testing of tacrolimus at 5 ng/mL

| | | | Mean | SD | Mean | | |
|---|---|---|---|---|---|---|---|
| | Drug | | reference | reference | test | SD test | Cross- |
| | Concentration | | sample | sample | sample | sample | reactivity |
| Drug tested | (ng/ml) | n | (ng/ml) | (ng/ml) | (ng/ml) | (ng/ml) | (ng/ml) |
| Sirolimus | 25 ng/ml | 6 | 5.07 | 0.49 | 5.23 | 0.85 | 0.17 |
| Cyclosporine | 300 ng/ml | 6 | 5.07 | 0.49 | 5.76 | 0.68 | 0.69 |

TABLE 5
Testing of tacrolimus at 20 ng/mL

| | | | Mean | SD | Mean | | |
|---|---|---|---|---|---|---|---|
| | Drug | | reference | reference | test | SD test | Cross- |
| | Concentration | | sample | sample | sample | sample | reactivity |
| Drug tested | (ng/ml) | n | (ng/ml) | (ng/ml) | (ng/ml) | (ng/ml) | (%) |
| Sirolimus | 25 ng/ml | 6 | 19.99 | 1.70 | 21.17 | 1.55 | 1.17 |
| Cyclosporine | 300 ng/ml | 6 | 19.99 | 1.70 | 19.79 | 1.97 | −0.20 |

Analog and metabolite cross reactivity was evaluated per the same protocol previously used for other immunosuppressant drugs. Drug analogs were spiked to reference whole blood samples containing 5 and 20 ng/mL Tac. The concentrations of drug analogs were tested at 20 ng/mL consistently (Table 6 and Table 7). The drug analogs tested were ascomycin, everolimus, pimecrolimus, ridaforolimus, temsirolimus, umirolimus, ascomycin, and zonotarolimus.

The cross-reactivity was calculated as the mean excess (i.e., false positive) Tac concentration (ng/mL) detected, based on the means of reference and tested sample. Significant cross reactivity would have been considered to be present when false positive readings exceeded three times the standard deviation of the tests, which was not observed for any of the tested analogs.

TABLE 6
Drug analogs tested with tacrolimus at 5 ng/mL

| | | | Mean | SD | Mean | | |
|---|---|---|---|---|---|---|---|
| | Drug | | Reference | Reference | Test | SD Test | Cross- |
| | Concentration | | Sample | Sample | Sample | Sample | reactivity |
| Drug Tested | (ng/mL) | n | (ng/mL) | (ng/mL) | (ng/mL) | (ng/mL) | (ng/mL) |
| Ascomycin | 5 | 6 | 6.04 | 0.72 | 9.62 | 0.84 | 3.58 |
| Everolimus | 5 | 6 | 6.04 | 0.72 | 5.30 | 0.52 | −0.74 |
| Pimecrolimus | 5 | 6 | 6.68 | 0.79 | 6.29 | 0.41 | −0.39 |
| Ridaforolimus | 5 | 6 | 6.68 | 0.79 | 5.50 | 0.37 | −1.18 |
| Temsirolimus | 5 | 6 | 5.67 | 0.73 | 5.30 | 0.51 | −0.38 |
| Umirolimus | 5 | 6 | 5.67 | 0.73 | 5.32 | 0.32 | −0.35 |
| Ascomycin | 5 | 6 | 7.74 | 0.79 | 11.58 | 1.23 | 3.84 |
| Zonotarolimus | 5 | 6 | 7.74 | 0.79 | 7.23 | 0.68 | −0.51 |

TABLE 7
Drug analogs tested with tacrolimus at 20 ng/mL

| | | | Mean | SD | Mean | | |
|---|---|---|---|---|---|---|---|
| | Drug | | Reference | Reference | Test | SD Test | Cross- |
| | Concentration | | Sample | Sample | Sample | Sample | reactivity |
| Drug Tested | (ng/mL) | n | (ng/mL) | (ng/mL) | (ng/mL) | (ng/mL) | (ng/mL) |
| Ascomycin | 20 | 6 | 24.83 | 3.13 | 32.15 | 3.29 | 7.32 |
| Everolimus | 20 | 6 | 24.83 | 3.13 | 22.06 | 0.96 | −2.77 |
| Pimecrolimus | 20 | 6 | 20.88 | 1.68 | 19.68 | 1.78 | −1.20 |
| Ridaforolimus | 20 | 6 | 20.88 | 1.68 | 20.03 | 0.99 | −0.84 |
| Temsirolimus | 20 | 6 | 18.25 | 1.62 | 19.80 | 2.68 | 1.55 |
| Umirolimus | 20 | 6 | 18.25 | 1.62 | 20.99 | 0.59 | 2.74 |
| Ascomycin | 20 | 6 | 20.22 | 1.24 | 26.08 | 2.76 | 5.86 |
| Zonotarolimus | 20 | 6 | 20.22 | 1.24 | 20.32 | 2.01 | 0.10 |

### Example 8: Calibration and Validation of the ELFA Platform

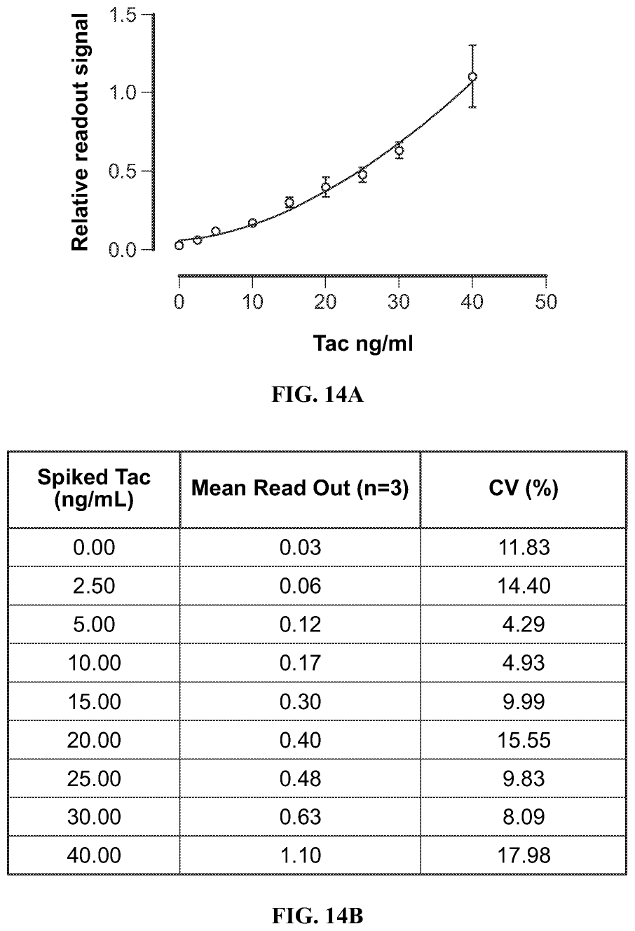

A calibration of the ELFA platform was conducted with plasma samples spiked with Tac in preparation for conducting the side-by-side testing against laboratory measurements for assay accuracy validation. Reference whole blood samples (negative for Tac) were procured from the commercial bio fluid source BioIVT and were aliquoted and spiked with Tac standard to a set of samples with concentrations of 0, 2.5, 5, 10, 15, 20, 25, 30, and 40 ng/mL. Samples were processed and tested following the described protocol in triplicate (n=3). Relative readout value of test line over control line was used as the dose response value to build the standard calibration curve (SCC). A four-parameter logistic model (4pl) built in GraphPad Prism was used to fit a curve to the data (FIGS. 14A-14B). The SCC showed a sensitivity of 2.5 ng/ml and a dynamic range of 0-40 ng/ml.

### Example 9: Comparison of Test Results from ELFA and Liquid Chromatograph-Mass Spectrometry (LCMS)

Two identical subsets of whole blood samples containing 0, 5, 10, 20, and 30 ng/ml of Tac were generated. One sample subset was sent to a certified standard laboratory for testing by liquid chromatography-mass spectrometry (LC-MS) analysis. The second subset was processed using the ELFA platform described herein, and the concentration was determined using a standard calibration curve. Each sample was tested with four replicates.

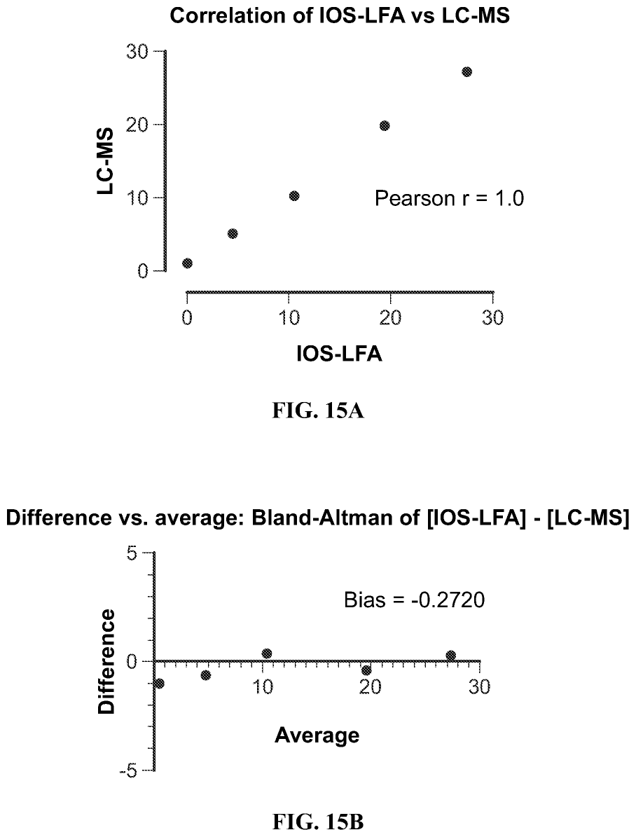

The concentrations of the samples were blinded to the LC-MS measurements. Results from the ELFA and laboratory LC-MS platforms are shown in Table 8. Results were then compared, using Pearson's correlation and Bland-Altman analysis (FIGS. 15A-15B). These results demonstrated a closely aligned correlation of the ELFA data and the LC-MS data, with Bland-Altman bias of −0.272 ng/mL.

TABLE 8
Results from ELFA and LC-MS platforms

| Spiked Tac | Tac Detected by ELFA | Tac Detected by LC-MS |
|---|---|---|
| (ng/mL) | (ng/mL) | (ng/mL) |
| 0.00 | 0.00 < 1 | ND |
| 5.00 | 4.49 | 5.10 |
| 10.00 | 10.57 | 10.20 |
| 20.00 | 19.40 | 19.80 |
| 30.00 | 27.48 | 27.20 |

ND, not determined.

### Example 10: Assessment of Intra- and Inter-Assay Repeatability

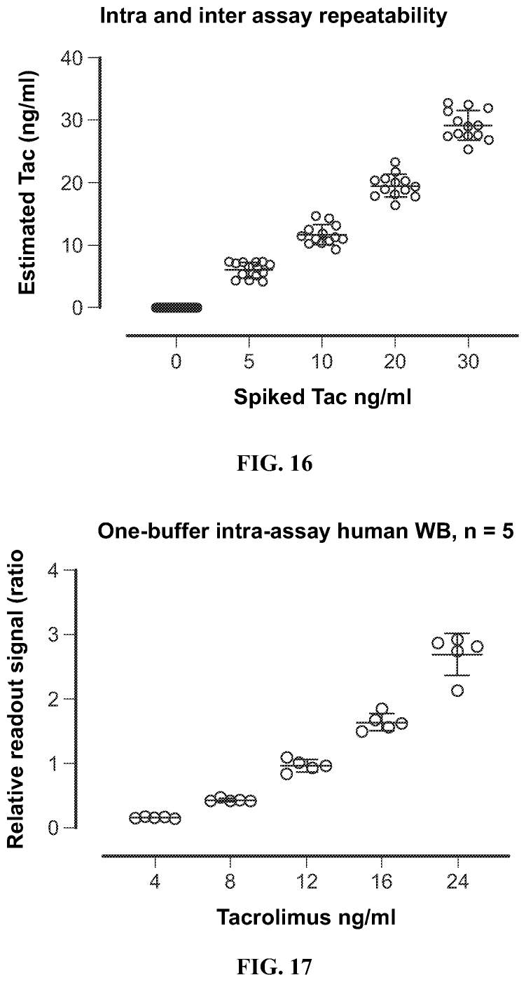

The intra-assay repeatability was assessed by CV (%) of replicates within the same test. The inter-assay repeatability was assessed by CV (%) of replicates from tests performed at different time points on the same sample. In total, four tests were run for a subset of blood samples spiked with 0, 5, 10, 20, and 30 ng/ml of Tac. Each test was performed with three replicates, with the exception of Test 1, which was run with four replicates. The concentration of each replicate was calculated from a test/control readout ratio and interpolated from the SCC seen in FIG. 16. The mean of each of the four tests is shown in Table 9. The average and CV of the four tests is considered the mean value and CV of the inter-assay test. The intra-and inter-assays have higher CVs at 5 ng/mL, but all levels tested were well separated.

TABLE 9
Intra- and inter-assay test data

| | Mean | | Mean | | Mean | | Mean | | | |
|---|---|---|---|---|---|---|---|---|---|---|
| Spiked | Test 1 | | Test 2 | | Test 3 | | Test 4 | | Average | |
| Tac | (n = 4) | Test 1 | (n = 3) | Test 2 | (n = 3) | Test 3 | (n = 3) | Test 4 | (Test 1-4) | Average |
| (ng/mL) | (ng/mL) | CV | (ng/mL) | CV | (ng/mL) | CV | (ng/mL) | CV | (ng/mL) | CV (%) |
| 0.00 | 0.00 | 0 | 0.00 | 0 | 0.00 | 0 | 0.00 | 0 | 0.00 | 0.00 |
| 5.00 | 4.49 | 14.36 | 6.55 | 18.39 | 6.72 | 13.73 | 7.10 | 5.83 | 6.21 | 18.85 |
| 10.00 | 10.57 | 8.76 | 13.06 | 9.75 | 12.78 | 13.92 | 10.74 | 4.68 | 11.79 | 11.15 |
| 20.00 | 19.40 | 11.45 | 18.67 | 6.54 | 19.30 | 6.88 | 20.88 | 10.83 | 19.56 | 4.78 |
| 30.00 | 27.48 | 5.62 | 30.99 | 9.75 | 30.36 | 7.85 | 28.54 | 5.32 | 29.34 | 5.51 |

### Example 11: Detection of Clinically Relevant Ranges of Tac Using an ELFA

The repeatability of the one-buffer assay was tested by performing intra-and inter-assay repeatability tests with human whole blood. Intra-assay testing was performed by spiking a sample of human whole blood from a single donor with Tac over a range of 4-24 ng/mL and testing the sample at five levels (4, 8, 12, 16, and 24 ng/ml). All samples at the same level were tested at the same time point. Repeatability was determined by the percent coefficient of variation (% CV), with the cutoff for satisfactory repeatability established at ≤10% CV. As seen in FIG. 17 and Table 10, the results showed good repeatability over the entire range.

TABLE 10
Results for intra-assay repeatability testing performed
on a single human whole blood sample spiked with Tac

| Spiked Tac | Average Readout | | |
|---|---|---|---|
| (ng/mL) | n = 5 | SD | % CV |
| 4 | 0.16 | 0.01 | 6% |
| 8 | 0.43 | 0.02 | 5% |
| 12 | 0.97 | 0.09 | 10% |
| 16 | 1.64 | 0.13 | 8% |
| 24 | 2.69 | 0.32 | 12% |

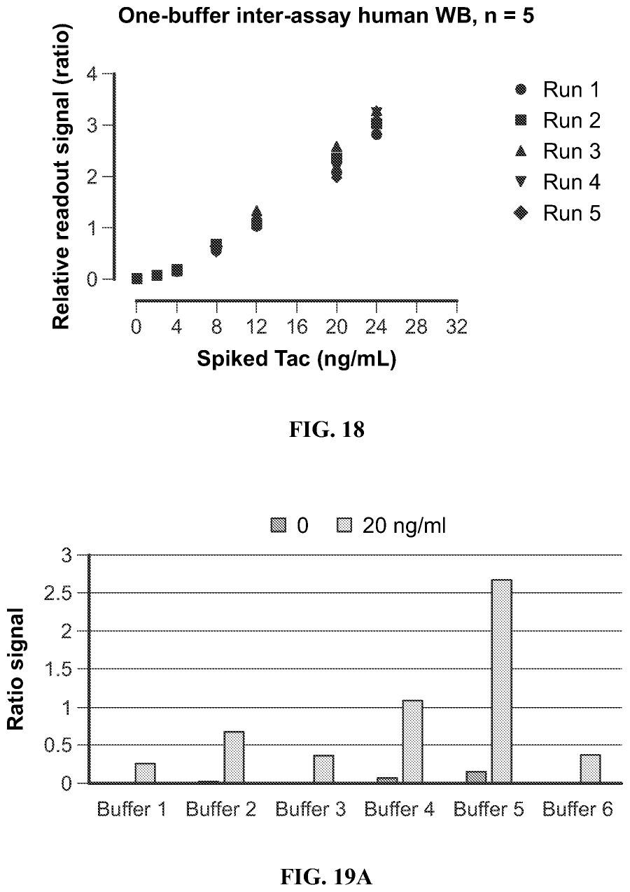

Repeatability was also assessed through inter-assay testing, in which a sample from a single donor was spiked with five levels of tacrolimus between 0 and 24 ng/ml. Each level was tested in one run at a single time point, with the testing repeated an additional four times for a total n=5. The % CV of each level was determined to assess the repeatability of the assay. As seen in FIG. 18 and Table 11, the results showed good repeatability, with % CV values near the 10% cutoff limit.

TABLE 11
Results for inter-assay repeatability testing performed
on a single human whole blood sample spiked with Tac

| Spiked | | | | | | | | |
|---|---|---|---|---|---|---|---|---|
| Tac | | | | | | | | |
| (ng/mL) | Run 1 | Run 2 | Run 3 | Run 4 | Run 5 | Mean | SD | CV (%) |
| 0 | 0.00 | 0.01 | 0.00 | 0.01 | 0.00 | 0.01 | 0.00 | ND |
| 2 | 0.08 | 0.08 | 0.07 | 0.08 | 0.09 | 0.08 | 0.01 | 7.56 |
| 4 | 0.15 | 0.16 | 0.19 | 0.21 | 0.16 | 0.17 | 0.02 | 13.39 |
| 8 | 0.54 | 0.68 | 0.66 | 0.54 | 0.59 | 0.60 | 0.06 | 10.43 |
| 12 | 1.11 | 1.07 | 1.30 | 1.18 | 1.04 | 1.14 | 0.10 | 9.14 |
| 20 | 2.07 | 2.37 | 2.58 | 2.17 | 1.99 | 2.24 | 0.24 | 10.74 |
| 24 | 2.82 | 3.02 | 3.28 | 3.24 | 3.12 | 3.09 | 0.18 | 5.96 |

### Example 12: Evaluating Various Sample Buffers for Use in the ELFA Platform

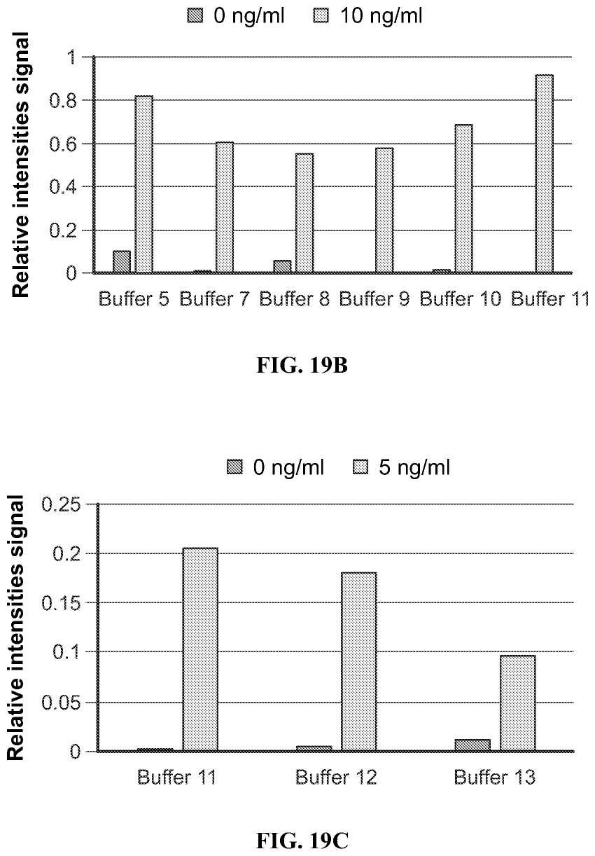

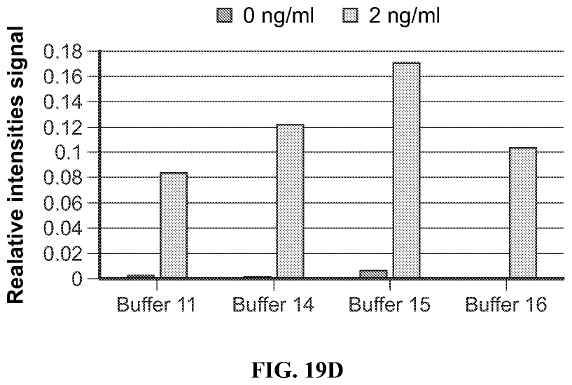

To identify sample buffers with high sensitivity and specificity for detection of tacrolimus, tests with various buffers using the one-buffer procedure were performed. Buffer compositions are shown in Table 12. The results showed high sensitivity and specificity for tacrolimus detection when buffer #5 was used in the ELFA platform (FIGS. 19A-19D). Buffers 7-11 were also evaluated and their performance was compared to Buffer 5 by testing blood samples spiked with 0 ng/mL and 10 ng/ml of tacrolimus. As shown in FIG. 19D, Buffer #15 showed high sensitivity and specificity, with a low negative control signal.

TABLE 12
Compositions of sample buffers

| | | | 4-(1,1,3,3- | | | | | |
|---|---|---|---|---|---|---|---|---|
| | | | Tetramethylbutyl) | potassium | | | | sodium |
| | | Polyoxyethylene | phenyl- | dihydrogen | potassium | sodium | trisodium | dihydrogen |
| | Polysorbate | (23) lauryl | polyethylene | phosphate | chloride | chloride | citrate | phosphate |
| BUFFER | 20 (% wt) | ether (% wt) | glycol (% wt) | (mM) | (mM) | (mM) | (mM) | (mM) |
| 1 | 0.05-1 | 0.5-2 | 0.05-1 | 1-5 | | 300-500 | 10-50 | 5-10 |
| 2 | 0.05-1 | 0.5-2 | 0.05-1 | 1-5 | | 300-500 | 10-50 | 5-10 |
| 3 | 0.05-1 | 0.5-2 | 0.05-1 | 1-5 | | 300-500 | 10-50 | 5-10 |
| 4 | 0.05-1 | 0.5-2 | 0.05-1 | 1-5 | | 300-500 | 10-50 | 5-10 |
| 5 | 0.05-1 | 0.5-2 | 0.05-1 | | | 600-1500 | 20-100 | |
| 6 | 0.5-1 | | 0.05-1 | 1-5 | 3-30 | 300-500 | | 5-10 |
| 7 | 0.05-1 | 0.5-2 | 0.05-1 | | | 600-1500 | 20-100 | |
| 8 | 0.05-1 | 0.5-2 | 0.05-1 | | | 600-1500 | 10-50 | |
| 9 | 0.05-1 | 0.5-2 | 0.05-1 | | | 600-1500 | 10-50 | |
| 10 | 0.05-1 | 0.5-2 | 0.05-1 | | | 300-500 | 45-50 | |
| 11 | 0.05-1 | 0.5-2 | 0.05-1 | | | 300-500 | 45-50 | |
| 12 | 0.05-1 | 0.5-2 | 0.05-1 | | | 300-500 | 45-50 | |
| 13 | 0.05-1 | 0.5-2 | 0.05-1 | | | 300-500 | 45-50 | |
| 14 | 0.05-1 | 0.5-2 | 0.05-1 | | | 300-500 | 45-50 | |
| 15 | 0.05-1 | 0.5-2 | 0.05-1 | | | 600-1500 | 20-100 | |
| 16 | 1-2 | 0.5-2 | 0.05-1 | | | 600-1500 | 20-100 | |

| | | | | | | sodium | | |
|---|---|---|---|---|---|---|---|---|
| | Polyethylene | | | | sodium | dodecyl | t- | |
| | glycol 20 k | trehalose | sucrose | casein | deoxycholate | sulfate | Octylphenoxypolyethoxyethanol | acetonitrile |
| BUFFER | (% wt) | (% wt) | (% wt) | (% wt) | (% wt) | (% wt) | (% wt) | (% wt) |
| 1 | 0.05-1 | 1-5 | 1-5 | 0.1-1 | | | | |
| 2 | 0.05-1 | 1-5 | 1-5 | 0.1-1 | 0.5-1 | 0.5-1 | | |
| 3 | 0.05-1 | 1-5 | 1-5 | 0.1-1 | | | 1-5 | |
| 4 | 0.05-1 | 1-5 | 1-5 | 0.1-1 | | 0.5-1 | | |
| 5 | 0.05-1 | 1-5 | 1-5 | 0.1-1 | 0.5-1 | 0.5-1 | | |
| 6 | 0.05-1 | 1-5 | 1-5 | 0.1-1 | 0.5-1 | 0.5-1 | | |
| 7 | 0.05-1 | 1-5 | 1-5 | 0.1-1 | | 0.5-1 | | |
| 8 | 0.05-1 | 1-5 | 1-5 | 0.1-1 | 0.5-1 | 0.5-1 | | |
| 9 | 0.05-1 | 1-5 | 1-5 | 0.1-1 | | | 0.5-1 | |
| 10 | 0.05-1 | 1-5 | 1-5 | 0.1-1 | | | | |
| 11 | 0.05-1 | 1-5 | 1-5 | 0.1-1 | 0.5-1 | 0.5-1 | | 5-30 |
| 12 | 0.05-1 | 1-5 | 1-5 | 0.1-1 | 0.5-1 | 1-2 | | 5-30 |
| 13 | 0.05-1 | 1-5 | 1-5 | 0.1-1 | 0.5-1 | | 1-2 | 5-30 |
| 14 | 0.05-1 | 1-5 | 1-5 | 0.1-1 | | | | 5-30 |
| 15 | 0.05-1 | 1-5 | 1-5 | 0.1-1 | | | | 5-30 |
| 16 | 0.05-1 | 1-5 | 1-5 | 0.1-1 | | | | 5-30 |

### OTHER EMBODIMENTS

As additional description to the embodiments described below, the present disclosure describes the following embodiments.

Embodiment 1 is a lateral flow device for detecting an analyte in a sample, the device comprising:
a sample region;
a conjugate region comprising:
   a detection agent that binds an analyte, wherein the detection agent is labeled with a first detectable label;
   a hybridization initiator coupled to the detection agent or the first detectable label;
   a plurality of nucleic acid probes, each of which is labeled with at least one second detectable label, wherein the plurality of nucleic acid probes comprises a first nucleic acid probe and a second nucleic acid probe, and a third nucleic acid probe;
      wherein the first nucleic acid probe hybridizes to a portion of the hybridization initiator and a portion of the second nucleic acid probe;
      wherein the second nucleic acid probe hybridizes to a portion of the first nucleic acid probe and a portion of the third nucleic acid probe; and
      wherein the third nucleic acid probe hybridizes to a portion of the second nucleic acid probe; and
a detection region comprising:
   a capture agent that binds the analyte, wherein the capture agent is immobilized on the detection region.

Embodiment 2 is the device of embodiment 1, wherein the detection agent comprises an antibody that binds the analyte.

Embodiment 3 is the device of embodiment 1 or embodiment 2, wherein a concentration of the detection agent applied on the conjugate region is in a range of 0.01 μg/μL to 0.1 μg/μL.

Embodiment 4 is the device of any one of embodiments 1-3, wherein the capture agent comprises an antibody that binds the analyte.

Embodiment 5 is the device of any one of embodiments 1-4, wherein a concentration of the capture agent applied on the detection region is in a range of 0.5 mg/mL to 5 mg/mL.

Embodiment 6 is the device of any one of embodiments 1-5, wherein the detection agent and the capture agent comprise the same antibody.

Embodiment 7 is the device of any one of embodiments 1-5, wherein the detection agent and the capture agent comprise different antibodies.

Embodiment 8 is the device of any one of embodiments 1-7, wherein the hybridization initiator comprises DNA, RNA, or both.

Embodiment 9 is the device of any one of embodiments 1-8, wherein a concentration of the hybridization initiator applied on the conjugate region is in a range of 5 nM to 40 nM.

Embodiment 10 is the device of any one of embodiments 1-9, wherein each nucleic acid probe in the plurality of nucleic acid probes comprises DNA, RNA, or both.

Embodiment 11 is the device of any one of embodiments 1-10, wherein a concentration of each nucleic acid probe in the plurality of nucleic acid probes applied on the conjugate region is in a range of 5 nM to 40 nM.

Embodiment 12 is the device of any one of embodiments 1-11, wherein the hybridization initiator and the first detectable label are conjugated to members of a receptor-ligand pair that mediates attachment of the hybridization initiator to the first detectable label.

Embodiment 13 is the device of any one of embodiments 1-12, wherein the nucleic acid probe and the second detectable label are conjugated to members of a receptor-ligand pair that mediates attachment of the nucleic acid probe to the second detectable label.

Embodiment 14 is the device of embodiment 12 or embodiment 13, wherein the receptor-ligand pair comprises biotin and avidin.

Embodiment 15 is the device of embodiment 14, wherein the avidin comprises streptavidin.

Embodiment 16 is the device of any one of embodiments 1-15, wherein the first detectable label and the second detectable label are visually detectable labels.

Embodiment 17 is the device of any one of embodiments 1-16, wherein the first detectable label comprises europium, colloidal gold, phycoerythrin, fluorescein, green fluorescent protein, quantum dots, rhodamine, or a combination thereof.

Embodiment 18 is the device of any one of embodiments 1-17, wherein the second detectable label comprises europium, colloidal gold, phycoerythrin, fluorescein, green fluorescent protein, quantum dots, rhodamine, or a combination thereof.

Embodiment 19 is the device of any one of embodiments 1-18, wherein the first detectable label and the second detectable label are the same.

Embodiment 20 is the device of any one of embodiments 1-19, wherein the first detectable label and the second detectable label comprise europium.

Embodiment 21 is the device of any one of embodiments 1-20, wherein the analyte is a drug.

Embodiment 22 is the device of embodiment 21, wherein the drug comprises an immunosuppressant, an antibiotic, an anticoagulant, an antidepressant, a bronchodilator, an anticonvulsant, an antiarrhythmic, an aminoglycoside, or a combination thereof.

Embodiment 23 is the device of embodiment 22, wherein the immunosuppressant comprises tacrolimus, ascomycin, everolimus, pimecrolimus, ridaforolimus, temsirolimus, umirolimus, ascomycin, sirolimus, cyclosporine, zonotarolimus, or a combination thereof.

Embodiment 24 is the device of any one of embodiments 1-23, wherein the detection region comprises a test region and a control region.

Embodiment 25 is the device of embodiment 24, wherein the test region comprises the capture agent and the control region comprises the control agent that binds to the detection agent.

Embodiment 26 is the device of any one of embodiments 1-25, further comprising an absorbent region for absorbing excess sample and maintaining a lateral flow along the detection region.

Embodiment 27 is the device of any one of embodiments 1-26, wherein the conjugate region is between the sample region and the detection region.

Embodiment 28 is the device of any one of embodiments 1-27, wherein the device is configured to allow the sample to flow from the sample region to the detection region.

Embodiment 29 is a sample buffer comprising:
a surfactant;
a salt;
a sugar; and
a protein.

Embodiment 30 is the buffer of embodiment 29, wherein the surfactant comprises sodium dodecyl sulfate, polysorbate, octylphenol ethoxylate, 2-[4-(2,4,4-trimethylpentan-2-yl)phenoxy]ethanol, sodium deoxycholate, 3-[N,N-Dimethyl(3-myristoylaminopropyl)ammonio]propanesulfonate, polyoxyethylene lauryl ether, polyethylene glycol, 4-(1,1,3,3-tetramethylbutyl)phenyl-polyethylene glycol, t-octylphenoxypolyethoxyethanol, or a combination thereof.

Embodiment 31 is the buffer of embodiment 29 or embodiment 30, wherein the salt comprises a sodium salt, a potassium salt, a phosphate salt, a zinc salt, or a combination thereof.

Embodiment 32 is the buffer of embodiment 31, wherein the sodium salt comprises sodium acetate, sodium carbonate, sodium bicarbonate, sodium chloride, sodium citrate, sodium phosphate monobasic, sodium phosphate dibasic, sodium sulfate, sodium thiosulfate, sodium triphosphate, or a combination thereof.

Embodiment 33 is the buffer of any one of embodiments 29-32, wherein the sugar comprises trehalose, sucrose, glucose, or a combination thereof.

Embodiment 34 is the buffer of any one of embodiments 29-33, wherein the protein comprises gelatin, albumin, casein, or a combination thereof.

Embodiment 35 is the buffer of any one of embodiments 29-34, wherein:
the surfactant comprises polysorbate 20, polyethylene glycol having an average molecular weight of 20,000 daltons (PEG 20), and 4-(1,1,3,3-tetramethylbutyl)phenyl-polyethylene glycol;
the sodium salt comprises sodium chloride;
the sugar comprises trehalose and sucrose; and
the protein comprises casein.

Embodiment 36 is the buffer of embodiment 35, wherein:
the polysorbate 20 is present in a total amount of about 0.05 wt % to about 10 wt % of the buffer;
the 4-(1,1,3,3-tetramethylbutyl)phenyl-polyethylene glycol is present in a total amount of about 0.001 wt % to about 4 wt % of the buffer;
the sodium chloride is present at a concentration of about 300 mM to about 1500 mM;
the PEG20 is present in a total amount of about 0.05 wt % to 10 wt % of the buffer;
the trehalose is present in a total amount of about 1 wt % to 30 wt % of the buffer;
the sucrose is present in a total amount of about 1 wt % to 30 wt % of the buffer; and
the casein is present in a total amount of about 0.1 wt % to 10 wt % of the buffer.

Embodiment 37 is the buffer of embodiment 35 or embodiment 36, wherein:
the polysorbate 20 is present in a total amount of about 0.05 wt % to about 2 wt % of the buffer;
the 4-(1,1,3,3-tetramethylbutyl)phenyl-polyethylene glycol is present in a total amount of about 0.05 wt % to about 1 wt % of the buffer;
the sodium chloride is present at a concentration of about 2 mM to about 500 mM;
the PEG20 is present in a total amount of about 0.05 wt % to 1 wt % of the buffer;
the trehalose is present in a total amount of about 1 wt % to 5 wt % of the buffer;
the sucrose is present in a total amount of about 1 wt % to 5 wt % of the buffer; and
the casein is present in a total amount of about 0.1 wt % to 1 wt % of the buffer.

Embodiment 38 is the buffer of any one of embodiments 29-37, further comprising polyoxyethylene (23) lauryl ether.

Embodiment 39 is the buffer of embodiment 38, wherein the polyoxyethylene (23) lauryl ether is present in a total amount of about 0.001 wt % to about 5 wt % of the buffer.

Embodiment 40 is the buffer of embodiment 38 or embodiment 39, wherein the polyoxyethylene (23) lauryl ether is present in a total amount of about 0.5 wt % to about 2 wt % of the buffer.

Embodiment 41 is the buffer of any one of embodiments 29-40, further comprising potassium dihydrogen phosphate.

Embodiment 42 is the buffer of embodiment 41, wherein the potassium dihydrogen phosphate is present at a concentration of about 1 mM to about 500 mM.

Embodiment 43 is the buffer of embodiment 41 or embodiment 42, wherein the potassium dihydrogen phosphate is present at a concentration of about 1 mM to about 5 mM.

Embodiment 44 is the buffer of any one of embodiments 29-43, further comprising potassium chloride.

Embodiment 45 is the buffer of embodiment 44, wherein the potassium chloride is present at a concentration of about 1 mM to about 200 mM.

Embodiment 46 is the buffer of embodiment 44 or embodiment 45, wherein the potassium chloride is present at a concentration of about 3 mM to about 30 mM.

Embodiment 47 is the buffer of any one of embodiments 29-46, further comprising trisodium citrate.

Embodiment 48 is the buffer of embodiment 47, wherein the trisodium citrate is present at a concentration of about 5 mM to about 500 mM.

Embodiment 49 is the buffer of embodiment 47 or embodiment 48, wherein the trisodium citrate is present at a concentration of about 10 mM to about 100 mM.

Embodiment 50 is the buffer of any one of embodiments 29-49, further comprising sodium dihydrogen phosphate.

Embodiment 51 is the buffer of embodiment 50, wherein the sodium dihydrogen phosphate is present at a concentration of about 1 mM to about 100 mM.

Embodiment 52 is the buffer of embodiment 50 or embodiment 51, wherein the sodium dihydrogen phosphate is present at a concentration of about 5 mM to about 10 mM.

Embodiment 53 is the buffer of any one of embodiments 29-52, further comprising sodium deoxycholate.

Embodiment 54 is the buffer of embodiment 53, wherein the sodium deoxycholate is present in a total amount of about 0.02 wt % to about 15 wt % of the buffer.

Embodiment 55 is the buffer of embodiment 53 or embodiment 54, wherein the sodium deoxycholate is present in a total amount of about 0.5 wt % to about 1 wt % of the buffer.

Embodiment 56 is the buffer of any one of embodiments 29-55, further comprising sodium dodecyl sulfate.

Embodiment 57 is the buffer of embodiment 56, wherein the sodium dodecyl sulfate is present in a total amount of about 0.5 wt % to about 5 wt % of the buffer.

Embodiment 58 is the buffer of embodiment 56 or embodiment 57, wherein sodium dodecyl sulfate is present in a total amount of about 0.5 wt % to about 2 wt % of the buffer.

Embodiment 59 is the buffer of any one of embodiments 29-58, further comprising t-octylphenoxypolyethoxyethanol.

Embodiment 60 is the buffer of embodiment 59, wherein the t-octylphenoxypolyethoxyethanol is present in a total amount of about 0.001 wt % to about 4 wt % of the buffer.

Embodiment 61 is the buffer of embodiment 59 or embodiment 60, wherein t-octylphenoxypolyethoxyethanol is present in a total amount of about 0.5 wt % to about 2 wt % of the buffer.

Embodiment 62 is the buffer of any one of embodiments 29-61, further comprising an organic solvent.

Embodiment 63 is the buffer of embodiment 62, wherein the organic solvent comprises methanol, acetone, acetonitrile, isopropanol, dichloromethane, ethanol, or a combination thereof.

Embodiment 64 is the buffer of embodiment 62 or embodiment 63, wherein the organic solvent comprises acetonitrile.

Embodiment 65 is the buffer of embodiment 63 or embodiment 64, wherein the acetonitrile is present in a total amount of about 1 wt % to about 50 wt % of the buffer.

Embodiment 66 is the buffer of any one of embodiments 63-65, wherein the acetonitrile is present in a total amount of about 5 wt % to about 30 wt % of the buffer.

Embodiment 67 is a method for detecting an analyte in a sample, the method comprising:
contacting the sample region of the device of any one of embodiments 1-28 with a sample;
transporting at least a portion of the sample through the conjugate region and the detection region via capillary flow;
binding an analyte to the detection agent bound to the first detectable label and at least one second detectable label to produce a complex comprising the detection agent and the analyte;
binding the complex comprising the detection agent and the analyte to the capture agent; and
detecting a signal from the first detectable label and the at least one second detectable label in the detection region.

Embodiment 68 is the method of embodiment 67, further comprising determining a level of the analyte in the sample based on the level of the signal.

Embodiment 69 is the method of embodiment 67 or embodiment 68, wherein detecting the signal is performed by a human eye.

Embodiment 70 is the method of embodiment 67 or embodiment 68, wherein detecting the signal is performed by placing the device in a signal detection reader.

Embodiment 71 is the method of any one of embodiments 67-70, further comprising combining the sample with the sample buffer of any one of embodiments 29-66 prior to contacting the sample with the device.

Embodiment 72 is the method of embodiment 71, wherein the sample comprises cells and the sample is incubated with the sample buffer for a time sufficient to release the analyte from the cells.

Embodiment 73 is the method of embodiment 71 or embodiment 72, wherein the sample is incubated with the sample buffer for 1 to 15 minutes prior to contacting the sample region with the sample.

Embodiment 74 is the method of any one of embodiments 67-73, wherein the sample is a biological sample obtained from a subject.

Embodiment 75 is the method of embodiment 74, wherein the biological sample is a whole blood sample, a serum sample, or a plasma sample.

Embodiment 76 is the method of embodiment 74 or embodiment 75, wherein the biological sample is a whole blood sample collected via a fingerstick device.

Embodiment 77 is the method of embodiment 76, wherein a volume of the whole blood sample is in a range of 5 μL to 100 μL.

Embodiment 78 is the method of any one of embodiments 74-77, wherein the biological sample is obtained by the subject.

Embodiment 79 is the method of any one of embodiments 74-78, wherein the subject has had or is going to have a tissue or organ transplant.

Embodiment 80 is the method of any one of embodiments 74-79, wherein the subject is a human patient.

Embodiment 81 is the method of any one of embodiments 74-80, wherein the analyte is a drug.

Embodiment 82 is the method of embodiment 81, wherein the drug comprises an immunosuppressant, an antibiotic, an anticoagulant, an antidepressant, a bronchodilator, an anticonvulsant, an antiarrhythmic, an aminoglycoside, or a combination thereof.

Embodiment 83 is the method of embodiment 82, wherein the immunosuppressant comprises tacrolimus, ascomycin, everolimus, pimecrolimus, ridaforolimus, temsirolimus, umirolimus, ascomycin, sirolimus, cyclosporine, zonotarolimus, or a combination thereof.

Embodiment 84 is a method of selecting a dose of an immunosuppressant for a subject, the method comprising:
obtaining a blood sample from a subject who has been administered an immunosuppressant at a first dose;
providing the blood sample to the sample region of any of the devices of embodiments 1-28;
advancing at least a portion of the blood sample through the conjugate region and the detection region via capillary flow;
binding an immunosuppressant to the detection agent coupled to the first detectable label and at least one second detectable label to produce a complex comprising the detection agent and the immunosuppressant;
binding the complex comprising the detection agent and the immunosuppressant to the capture agent; and
detecting a signal from the first detectable label and the at least one second detectable label in the detection region.

Embodiment 85 is the method of embodiment 84, further comprising determining a level of the immunosuppressant in the sample based on the level of the signal.

Embodiment 86 is the method of embodiment 84 or embodiment 85, further comprising determining a second dose of the immunosuppressant for the subject based on the level of the immunosuppressant in the sample.

Embodiment 87 is the method of any one of embodiments 84-86, wherein obtaining the blood sample comprises using a fingerstick device.

Embodiment 88 is the method of any one of embodiments 84-87, wherein contacting the blood sample with the sample region comprises applying the blood directly to the sample region.

Embodiment 89 is the method of any one of embodiments 84-88, further comprising combining the blood sample with any one of the sample buffers of embodiment 29-66 prior to contacting the blood sample with the device.

Embodiment 90 is the method of embodiment 89, wherein the blood sample is incubated with the sample buffer for a time sufficient to release the immunosuppressant from the blood cells.

Embodiment 91 is the method of embodiment 89 or embodiment 90, wherein the blood sample is incubated with the sample buffer for 1 to 15 minutes prior to contacting the blood sample with the device.

Embodiment 92 is the method of any one of embodiments 84-88, further comprising administering the immunosuppressant at a second dose based on the amount of the immunosuppressant in the blood sample.

Embodiment 93 is the method of embodiment 92, wherein the second dose is higher or lower than the first dose.

Embodiment 94 is a kit comprising:
any one of the devices of embodiments 1-28; and
any one of the sample buffers of embodiments 29-66.

Embodiment 95 is the kit of embodiment 94, further comprising a fingerstick device.

Embodiment 96 is the kit of embodiment 94 or embodiment 95, further comprising a signal detection reader.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. A lateral flow device for detecting an analyte in a sample, the device comprising:
a sample region;
a conjugate region comprising:
a detection agent that binds an analyte, wherein the detection agent is labeled with a first detectable label;
a hybridization initiator coupled to the detection agent or the first detectable label;
a plurality of nucleic acid probes, each of which is labeled with at least one second detectable label, wherein the plurality of nucleic acid probes comprises a first nucleic acid probe and a second nucleic acid probe, and a third nucleic acid probe;
wherein the first nucleic acid probe hybridizes to a portion of the hybridization initiator and a portion of the second nucleic acid probe;
wherein the second nucleic acid probe hybridizes to a portion of the first nucleic acid probe and a portion of the third nucleic acid probe; and
wherein the third nucleic acid probe hybridizes to a portion of the second nucleic acid probe; and
a detection region comprising:
a capture agent that binds the analyte, wherein the capture agent is immobilized on the detection region.

2. The device of claim 1, wherein the detection agent comprises an antibody that binds the analyte.

3. The device of claim 1, wherein a concentration of the detection agent applied on the conjugate region is in a range of 0.01 μg/μL to 0.1 μg/μL.

4. The device of claim 1, wherein the capture agent comprises an antibody that binds the analyte.

5. The device of claim 1, wherein a concentration of the capture agent applied on the detection region is in a range of 0.5 mg/mL to 5 mg/mL.

6. The device of claim 1, wherein the detection agent and the capture agent comprise the same antibody.

7. The device of claim 1, wherein the detection agent and the capture agent comprise different antibodies.

8. The device of claim 1, wherein the hybridization initiator comprises DNA, RNA, or both.

9. The device of claim 1, wherein a concentration of the hybridization initiator applied on the conjugate region is in a range of 5 nM to 40 nM.

10. The device of claim 1, wherein each nucleic acid probe in the plurality of nucleic acid probes comprises DNA, RNA, or both.

11. The device of claim 1, wherein a concentration of each nucleic acid probe in the plurality of nucleic acid probes applied on the conjugate region is in a range of 5 nM to 40 nM.

12. The device of claim 1, wherein the hybridization initiator and the first detectable label are conjugated to members of a receptor-ligand pair that mediates attachment of the hybridization initiator to the first detectable label.

13. The device of claim 1, wherein the nucleic acid probe and the second detectable label are conjugated to members of a receptor-ligand pair that mediates attachment of the nucleic acid probe to the second detectable label.

14. The device of claim 12, wherein the receptor-ligand pair comprises biotin and avidin.

15. The device of claim 14, wherein the avidin comprises streptavidin.

16. The device of claim 1, wherein the first detectable label and the second detectable label are visually detectable labels.

17. The device of claim 1, wherein the first detectable label comprises europium, colloidal gold, phycoerythrin, fluorescein, green fluorescent protein, quantum dots, rhodamine, or a combination thereof.

18. The device of claim 1, wherein the second detectable label comprises europium, colloidal gold, phycoerythrin, fluorescein, green fluorescent protein, quantum dots, rhodamine, or a combination thereof.

19. The device of claim 1, wherein the first detectable label and the second detectable label are the same.

20. The device of claim 1, wherein the first detectable label and the second detectable label comprise europium.

21. The device of claim 1, wherein the analyte is a drug.

22. The device of claim 21, wherein the drug comprises an immunosuppressant, an antibiotic, an anticoagulant, an antidepressant, a bronchodilator, an anticonvulsant, an antiarrhythmic, an aminoglycoside, or a combination thereof.

23. The device of claim 22, wherein the immunosuppressant comprises tacrolimus, ascomycin, everolimus, pimecrolimus, ridaforolimus, temsirolimus, umirolimus, ascomycin, sirolimus, cyclosporine, zonotarolimus, or a combination thereof.

24. The device of claim 1, wherein the detection region comprises a test region and a control region.

25. The device of claim 24, wherein the test region comprises the capture agent and the control region comprises the control agent that binds to the detection agent.

26. The device of claim 1, further comprising an absorbent region for absorbing excess sample and maintaining a lateral flow along the detection region.

27. The device of claim 1, wherein the conjugate region is between the sample region and the detection region.

28. The device of claim 1, wherein the device is configured to allow the sample to flow from the sample region to the detection region.

29. A sample buffer comprising:
a surfactant;
a salt;
a sugar; and
a protein.

30. The buffer of claim 29, wherein the surfactant comprises sodium dodecyl sulfate, polysorbate, octylphenol ethoxylate, 2-[4-(2,4,4-trimethylpentan-2-yl)phenoxy]ethanol, sodium deoxycholate, 3-[N,N-Dimethyl(3-myristoylaminopropyl)ammonio]propanesulfonate, polyoxyethylene lauryl ether, polyethylene glycol, 4-(1,1,3,3-tetramethylbutyl)phenyl-polyethylene glycol, t-octylphenoxypolyethoxyethanol, or a combination thereof.

31. The buffer of claim 29, wherein the salt comprises a sodium salt, a potassium salt, a phosphate salt, a zinc salt, or a combination thereof.

32. The buffer of claim 31, wherein the sodium salt comprises sodium acetate, sodium carbonate, sodium bicarbonate, sodium chloride, sodium citrate, sodium phosphate monobasic, sodium phosphate dibasic, sodium sulfate, sodium thiosulfate, sodium triphosphate, or a combination thereof.

33. The buffer of claim 29, wherein the sugar comprises trehalose, sucrose, glucose, or a combination thereof.

34. The buffer of claim 29, wherein the protein comprises gelatin, albumin, casein, or a combination thereof.

35. The buffer of claim 29, wherein:
the surfactant comprises polysorbate 20, polyethylene glycol having an average molecular weight of 20,000 daltons (PEG 20), and 4-(1,1,3,3-tetramethylbutyl)phenyl-polyethylene glycol;
the sodium salt comprises sodium chloride;
the sugar comprises trehalose and sucrose; and
the protein comprises casein.

36. The buffer of claim 35, wherein:
the polysorbate 20 is present in a total amount of about 0.05 wt % to about 10 wt % of the buffer;
the 4-(1,1,3,3-tetramethylbutyl)phenyl-polyethylene glycol is present in a total amount of about 0.001 wt % to about 4 wt % of the buffer;
the sodium chloride is present at a concentration of about 300 mM to about 1500 mM;
the PEG20 is present in a total amount of about 0.05 wt % to 10 wt % of the buffer;
the trehalose is present in a total amount of about 1 wt % to 30 wt % of the buffer;
the sucrose is present in a total amount of about 1 wt % to 30 wt % of the buffer; and
the casein is present in a total amount of about 0.1 wt % to 10 wt % of the buffer.

37. The buffer of claim 36, wherein:
the polysorbate 20 is present in a total amount of about 0.05 wt % to about 2 wt % of the buffer;
the 4-(1,1,3,3-tetramethylbutyl)phenyl-polyethylene glycol is present in a total amount of about 0.05 wt % to about 1 wt % of the buffer;
the sodium chloride is present at a concentration of about 2 mM to about 500 mM;
the PEG20 is present in a total amount of about 0.05 wt % to 1 wt % of the buffer;
the trehalose is present in a total amount of about 1 wt % to 5 wt % of the buffer;
the sucrose is present in a total amount of about 1 wt % to 5 wt % of the buffer; and
the casein is present in a total amount of about 0.1 wt % to 1 wt % of the buffer.

38. The buffer of claim 29, further comprising polyoxyethylene (23) lauryl ether.

39. The buffer of claim 38, wherein the polyoxyethylene (23) lauryl ether is present in a total amount of about 0.001 wt % to about 5 wt % of the buffer.

40. The buffer of claim 39, wherein the polyoxyethylene (23) lauryl ether is present in a total amount of about 0.5 wt % to about 2 wt % of the buffer.

41. The buffer of claim 29, further comprising potassium dihydrogen phosphate.

42. The buffer of claim 41, wherein the potassium dihydrogen phosphate is present at a concentration of about 1 mM to about 500 mM.

43. The buffer of claim 42, wherein the potassium dihydrogen phosphate is present at a concentration of about 1 mM to about 5 mM.

44. The buffer of claim 29, further comprising potassium chloride.

45. The buffer of claim 44, wherein the potassium chloride is present at a concentration of about 1 mM to about 200 mM.

46. The buffer of claim 45, wherein the potassium chloride is present at a concentration of about 3 mM to about 30 mM.

47. The buffer of claim 29, further comprising trisodium citrate.

48. The buffer of claim 47, wherein the trisodium citrate is present at a concentration of about 5 mM to about 500 mM.

49. The buffer of claim 48, wherein the trisodium citrate is present at a concentration of about 10 mM to about 100 mM.

50. The buffer of claim 29, further comprising sodium dihydrogen phosphate.

51. The buffer of claim 50, wherein the sodium dihydrogen phosphate is present at a concentration of about 1 mM to about 100 mM.

52. The buffer of claim 51, wherein the sodium dihydrogen phosphate is present at a concentration of about 5 mM to about 10 mM.

53. The buffer of claim 29, further comprising sodium deoxycholate.

54. The buffer of claim 53, wherein the sodium deoxycholate is present in a total amount of about 0.02 wt % to about 15 wt % of the buffer.

55. The buffer of claim 54, wherein the sodium deoxycholate is present in a total amount of about 0.5 wt % to about 1 wt % of the buffer.

56. The buffer of claim 29, further comprising sodium dodecyl sulfate.

57. The buffer of claim 56, wherein the sodium dodecyl sulfate is present in a total amount of about 0.5 wt % to about 5 wt % of the buffer.

58. The buffer of claim 57, wherein sodium dodecyl sulfate is present in a total amount of about 0.5 wt % to about 2 wt % of the buffer.

59. The buffer of claim 29, further comprising t-octylphenoxypolyethoxyethanol.

60. The buffer of claim 59, wherein the t-octylphenoxypolyethoxyethanol is present in a total amount of about 0.001 wt % to about 4 wt % of the buffer.

61. The buffer of claim 60, wherein t-octylphenoxypolyethoxyethanol is present in a total amount of about 0.5 wt % to about 2 wt % of the buffer.

62. The buffer of claim 29, further comprising an organic solvent.

63. The buffer of claim 62, wherein the organic solvent comprises methanol, acetone, acetonitrile, isopropanol, dichloromethane, ethanol, or a combination thereof.

64. The buffer of claim 63, wherein the organic solvent comprises acetonitrile.

65. The buffer of claim 64, wherein the acetonitrile is present in a total amount of about 1 wt % to about 50 wt % of the buffer.

66. The buffer of claim 63, wherein the acetonitrile is present in a total amount of about 5 wt % to about 30 wt % of the buffer.

67. A method for detecting an analyte in a sample, the method comprising:
contacting the sample region of the device of claim 1 with a sample;
transporting at least a portion of the sample through the conjugate region and the detection region via capillary flow;
binding an analyte to the detection agent bound to the first detectable label and at least one second detectable label to produce a complex comprising the detection agent and the analyte;
binding the complex comprising the detection agent and the analyte to the capture agent; and
detecting a signal from the first detectable label and the at least one second detectable label in the detection region.

68. The method of claim 67, further comprising determining a level of the analyte in the sample based on the level of the signal.

69. The method of claim 67, wherein detecting the signal is performed by a human eye.

70. The method of claim 67, wherein detecting the signal is performed by placing the device in a signal detection reader.

71. The method of claim 67 further comprising combining the sample with the sample buffer of claim 29 prior to contacting the sample with the device.

72. The method of claim 71, wherein the sample comprises cells and the sample is incubated with the sample buffer for a time sufficient to release the analyte from the cells.

73. The method of claim 72, wherein the sample is incubated with the sample buffer for 1 to 15 minutes prior to contacting the sample region with the sample.

74. The method of claim 67, wherein the sample is a biological sample obtained from a subject.

75. The method of claim 74, wherein the biological sample is a whole blood sample, a serum sample, or a plasma sample.

76. The method of claim 75, wherein the biological sample is a whole blood sample collected via a fingerstick device.

77. The method of claim 76, wherein a volume of the whole blood sample is in a range of 5 μL to 100 μL.

78. The method of claim 74, wherein the biological sample is obtained by the subject.

79. The method of claim 74, wherein the subject has had or is going to have a tissue or organ transplant.

80. The method of claim 74, wherein the subject is a human patient.

81. The method of claim 74, wherein the analyte is a drug.

82. The method of claim 81, wherein the drug comprises an immunosuppressant, an antibiotic, an anticoagulant, an antidepressant, a bronchodilator, an anticonvulsant, an antiarrhythmic, an aminoglycoside, or a combination thereof.

83. The method of claim 82, wherein the immunosuppressant comprises tacrolimus, ascomycin, everolimus, pimecrolimus, ridaforolimus, temsirolimus, umirolimus, ascomycin, sirolimus, cyclosporine, zonotarolimus, or a combination thereof.

84. A method of selecting a dose of an immunosuppressant for a subject, the method comprising:
obtaining a blood sample from a subject who has been administered an immunosuppressant at a first dose;
providing the blood sample to the sample region of the device of claim 1;
advancing at least a portion of the blood sample through the conjugate region and the detection region via capillary flow;
binding an immunosuppressant to the detection agent coupled to the first detectable label and at least one second detectable label to produce a complex comprising the detection agent and the immunosuppressant;
binding the complex comprising the detection agent and the immunosuppressant to the capture agent; and
detecting a signal from the first detectable label and the at least one second detectable label in the detection region.

85. The method of claim 84, further comprising determining a level of the immunosuppressant in the sample based on the level of the signal.

86. The method of claim 84, further comprising determining a second dose of the immunosuppressant for the subject based on the level of the immunosuppressant in the sample.

87. The method of claim 84, wherein obtaining the blood sample comprises using a fingerstick device.

88. The method of claim 84, wherein contacting the blood sample with the sample region comprises applying the blood directly to the sample region.

89. The method of claim 84, further comprising combining the blood sample with any one of the sample buffers of claim 29 prior to contacting the blood sample with the device.

90. The method of claim 89, wherein the blood sample is incubated with the sample buffer for a time sufficient to release the immunosuppressant from the blood cells.

91. The method of claim 90, wherein the blood sample is incubated with the sample buffer for 1 to 15 minutes prior to contacting the blood sample with the device.

92. The method of claim 84, further comprising administering the immunosuppressant at a second dose based on the amount of the immunosuppressant in the blood sample.

93. The method of claim 92, wherein the second dose is higher or lower than the first dose.

94. A kit comprising:
the device of claim 1; and
a sample buffer comprising:
a surfactant;
a salt;
a sugar; and
a protein.

95. The kit of claim 94, further comprising a fingerstick device.

96. The kit of claim 94, further comprising a signal detection reader.
